(19) United States
(12) Patent Application Publication
(10) Pub. No.: US 2006/0172427 A1
(43) Pub. Date: Aug. 3, 2006

(54) Method and apparatus for retrofitting existing real time control systems for monitoring, controlling, and distributing chemicals during electroplating

(76) Inventors: Omar Germouni, Chicago, IL (US); Alan D. Zdunek, Chicago, IL (US)

Correspondence Address: AIR LIQUIDE, 2700 POST OAK BOULEVARD, SUITE 1800, HOUSTON, TX, 77056, US

(21) Appl. No.: 11/296,067

(22) Filed: Dec. 7, 2005

Related U.S. Application Data
(63) Continuation-in-part of application No. 10/829,596, filed on Apr. 21, 2004, now Pat. No. 6,937,063.
(60) Provisional application No. 60/465,184, filed on Apr. 23, 2003.
(60) Provisional application No. 60/542,741, filed on Feb. 5, 2004.
(60) Provisional application No. 60/476,931, filed on Jun. 9, 2003.
(60) Provisional application No. 60/556,864, filed on Mar. 26, 2004.

(51) Int. Cl. G01N 33/00

(52) U.S. Cl. 436/55

(57) ABSTRACT

Chemical treatment process control, including a virtual sensor based upon artificial intelligence that automatically computes and predicts additive concentrations in chemical baths used in manufacturing to compensate for the lag time in obtaining data from real-time analyzers (RTA). Once actual measurement data is obtained, bath concentrations can be further adjusted if necessary. Also disclosed is the retrofitting of a RTA with a controller where the data signal from the RTA has a proprietary communication protocol that is converted by hardware and/or software into a signal having an open communication protocol for transmission to the controller for controlling electrochemical bath concentration. Further disclosed is a method of controlling chemical concentration of electrochemical baths by predicting the depletion of chemicals during manufacture and causing dosing and/or draining of a portion of the bath during the time delay between RTA analyses. An algorithm written into program language and compiled into an executable format can be used in the controllers.

### CROSS-REFERENCE TO RELATED APPLICATIONS

This application is a continuation in part application of U.S. patent application Ser. No. 10/827,596, filed Apr. 19, 2004, and entitled Method And Apparatus for Monitoring, Dosing And Distribution Of Chemical Solutions, which is related to and claims priority to U.S. provisional patent application Ser. No. 60/465,184 filed on Apr. 23,2003, 60/542,741, filed on Feb. 5, 2004, 60/476,931 filed on Jun. 9, 2003, and 60/556,864 filed on Mar. 26, 2004, the entire contents of which are incorporated by reference.

### BACKGROUND

Many material treatment processes utilize a chemical composition. In order to smoothly operate such a process, upsets or deviations in the quality of the chemical composition should be avoided. Otherwise, quality of the treated material will vary over time. Also, the process might need to be shut down in order to bring the quality of the chemical composition back into the specification of the process. Such a shutdown is often prohibitively expensive.

Some examples of material treatment processes include chemical mechanical polishing/planarization (CMP), electrochemical polishing/planarization (ECP), or copper electroplating (ECD) of semiconductor wafers or coated semiconductor wafers.

Regarding ECD, achieving defect-free copper interconnects on integrated circuits by electroplating has involved the development of a new process called “superfilling” or “bottom-up plating”. Key parts in this process are the organic additives used in the plating bath. These critical chemical components, called brighteners, suppressors, and levelers, have specifically been tailored to promote the critical bottom-up plating or superconformal deposition that allows high-aspect ratio trenches and vias to be filled correctly. Furthermore, since these additives become depleted during the deposition process, bath monitoring, dosing and solution delivery are critical to process stability, uniform deposition rate, and obtaining the correct physical properties of the copper layer (such as morphology, microstructure, conductivity and grain size).

Presently, copper bath monitoring is performed by measuring the composition of the inorganic and organic components in the bath solution contained in the reservoir tank of the process tool. This is done commercially by offline or online methods:

Methods of offline measurements used to analyze bath composition include titration, high performance liquid chromatography (HPLC), cyclic voltammetric stripping (CVS), and modified CVS. Titration is used to measure the inorganic components such as copper, acidity and chloride. CVS and HPLC are used to measure the concentration of the organic additives, the brightener, suppressor and leveler. With respect to CVS, ECI Technology has commercial equipment based on this technique [Bratin P., Chalyt G., Pavlov M., “Control of Damascene Copper Processes by Cyclic Voltammetric Stripping, Plating & Surface Finishing, March 2000, pp. 14-16], which is hereby fully incorporated herein by reference. U.S. Pat. Nos. 6,365,033 and 6,551,479 also disclose pulsed voltammetric stripping, and are hereby fully incorporated herein by reference. With respect to HPLC, Dionex discloses this technique [Dionex Industry Brief; “Analysis of Copper Plating Baths”, pp. 1-8], as does U.S. Pat. No. 6,740,242, entitled “Plating Apparatus and Method of Managing Plating Liquid Composition”, both of which are incorporated herein by reference.

One of the main limitations of CVS is its sensitivity to matrix effects of the electrolyte, since the copper stripping is dependent not only on the individual additives but also their interaction and degradation in the bath solution [Sun, Zhi-Wen and Dixit, Girsih, “Optimized bath control for void-free copper deposition”, Solid State Technology, November, 2001, pp. 97-102; and Taylor, T., Ritzdorf, T., Lindberg F., Carpenter B., LeFebvre M., “Electroplating Bath Controls for Copper Interconnects”, Solid State Technology, November, 1998]. Also, measurement time tends to be long (several hours) and large volumes of electrolytes are used, increasing the consumption and waste stream of these expensive chemicals. High performance liquid chromatography (HPLC) also generates large amounts of waste and also has a relatively long response time.

Off-line systems, such as those sold by ECI Technology, are in fact fully automated analyzers that perform cyclic voltammetric stripping (CVS) on samples taken from the plating bath at preset time intervals. Based on analysis results, human intervention is required to input control parameters to the bath control system. Typically, it takes several hours for these off-line systems to provide additive concentrations for process control. This can result in the production of less than optimal, or even inferiorly plated items.

Methods of online measurements include periodic sampling of the bath and analysis at or near the plating tool to monitor bath component concentrations. One limitation to many of these methods is that plating bath breakdown products are not measured, although they are critical to determining bath quality. Some examples of online monitoring include the following. Bratin P., Chalyt G., Pavlov M., “Control of Damascene Copper Processes by Cyclic Voltammetric Stripping, Plating & Surface Finishing,” March 2000, pp. 14-16 disclose several types of online monitor methods that measure component concentration by titration and cyclic voltammetric stripping. A company named Technic, Inc. provides online AC/DC voltammetric monitoring, such as that disclosed in U.S. Pat. No. 5,755,954, entitled “Method of Monitoring Constituents in Electroless Plating Baths”, which is hereby fully incorporated herein by reference, and titration/pulsed cyclic galvanostatic analysis (ATMI). Also, a company named ATMI offers titration/pulsed cyclic galvanostatic analysis. As another example, U.S. Pat. No. 6,635,157 discloses online titration and CVS. As further examples, U.S. Pat. Nos. 6,365,157 and 6,551,749 disclose online CVS monitoring.

One of the key objectives electroplating bath management is to maintain concentrations in the baths at desired levels by consistently dosing additives and fresh solution (inorganic solution) to counterbalance depletion of the additives in the bath due to byproduct formation during plating, degradation, and/or solution bleeding.

Copper bath concentrations used in wafer plating tools are typically maintained in two ways: 1) open-loop control, which consists of “semi-automatic” dosing based on an empirical equation that relates depletion of a component with the number of wafers deposited (or amount of total current used), and 2) offline measurements, which include daily sampling and offline analysis with the results sent back to the process engineer who doses the plating bath accordingly to maintain it in optimal condition. Open-loop control generally works, but may fail to correct for local variations in consumption associated with plating parameter fluctuations or changing bath equilibrium as the bath solution ages.

Current state-of-the-art dosing and bleeding of the bath is performed at the reservoir tank of the process tool. For instance, the makeup electrolyte (inorganic components) is delivered from a sub-fab chemical delivery system to the process tool and dosed into the reservoir tank. The additives are stored in small containers in the process tool and are dosed directly into the reservoir tank. Bleeding of the bath is also performed at the reservoir tank. Dosing control is performed by two methods: open-loop and closed-loop controls.

With regard to open-loop control, U.S. Pat. No. 6,471,845 B1 discloses empirical equations used for dosage based on previously defined consumption rates of the various components, and which accounts for the number of wafers processed, the plating current density, and other factors. Novellus has been involved in attempts to close the control loops using monitoring techniques, such as mass spectrometry as disclosed in U.S. Pat. No. 6,726,824 entitled, “Closed Loop Monitoring of Electroplate Bath Constituents Using Mass Spectrometry,” which is hereby fully incorporated herein by reference, to identify bath conditions and predict its formulation. As another example, U.S. Pat. No. 6,458,262 B1 discloses a chemical consumption process model based on off-line or online measurements to achieve bath control. As an example, U.S. Pat. No. 5,352,350 discloses empirical equations used for online, open loop control of the bath concentrations. These models are tool, chemistry and production regime specific. By design, they can, at best, only control conditioned plating solution around well-known operating conditions.

Others recognize that open-loop control fails to correct for local variations in consumption associated with plating fluctuations or changing bath equilibrium as degradation products are formed. See for example, Bratin P et al., “Control of Damascene Copper Processes by Cyclic Voltammetric Stripping, Plating & Surface Finishing”, at pp. 14-16]. Also, these types of models are likely only valid in the vicinity of a known operating regime and have limited ability to accommodate process upsets or disturbances. Since open loop control does not detect process changes as it assumes steady-state operation, it could eventually lead to serious copper interconnects defects.

With regard to closed-loop control, bath sampling with either offline or online analysis is performed as described above. The results are sent back to the process engineer who doses the plating bath accordingly to maintain it in optimal condition. Offline or online analysis is used to “correct” for concentrations that drift due to incorrect open-loop control. However, current online bath component analysis is performed in 20-40 minutes depending on the type of equipment, which is not quite “real-time”. In addition, communication of the online monitor to the dosing system of commercial plating tools is not yet automatic and requires development of communication protocols to effectively use the process signal to control the bath concentrations. U.S. Pat. No. 6,592,736 provides one example of a closed-loop control.

As mentioned above, most commercial copper interconnect plating tools are stand-alone systems where the additives and makeup electrolyte are added individually to a reservoir tank located inside the plating tool and recirculated to and from the wafer plating cells. A portion of the bath solution in the reservoir tank can be bled out periodically to reduce contamination buildup. However, depending on wafer plating production levels, additive bottles may need to be changed quite frequently at the tool, which increases the risk of operator error and contamination (for example, from an incorrect bottle exchange).

Online monitoring for concentration control may be integrated into the tool or not. For those tools that do not integrate online monitoring with the tool, they are disadvantageous because they require process engineer intervention to correct the bath concentrations. Furthermore, most semiconductor fabs or foundries utilize more than one wafer-plating tool in production. Since each tool is controlled separately, inconsistencies in the electrochemical bath solution between tools can develop which can ultimately result in different properties of the copper interconnect deposit for different tools.

The trend is towards online monitoring since real-time control of the bath can theoretically be achieved, supplanting empirical consumption models that now control the dosing. For instance, “online monitoring” equipment is commercially available from ECI Technology and ATMI. However, the equipment performs “batch” analysis of electrochemical solutions pumped from the plating tool and thus is not truly an online system. Technic, Inc. has developed a second category of commercially available analyzers referred to as real-time analyzers. These systems are able to measure bath concentrations inside the plating tool and also within the hour due to the use electrochemical methods and complex interpolation algorithms that are based upon past processing conditions and analyses as predictors of parameters for operating conditions, which turn out to be accurate and much faster than conventional off-line analyzers.

In either case, existing off-line or on-line prior art analyzers are standalone systems in that they are not interfaced with a process controller or process control system. This prevents the closed loop, automatic feedback scheme sought in any process control system. In other words, human intervention is still needed to feedback concentration data to the main control system.

Real-time monitors also referred to herein as real time analyzers are often designed to run in local or standalone mode, in laboratory or in research environments, and on the plant floor as well. They are often programmed via proprietary user interfaces that run on office-type or Windows® (Microsoft Corp., Redmond Wash.) or Windows®-type Operating Systems.

When remote control is needed for real-time feedback control, it is necessary to achieve connectivity between the standalone device and the main process control system. The difficulty is to seamlessly interface to these different operating systems and their specific applications using reliable, easy to implement and a cost effective industrial network for process control. Finally, in a non-limiting embodiment, the retrofit integration does not require substantial modification of the hardware or software on either system making it even faster to retrofit.

In a specific case, the real-time analyzer (RTA) by Technic proved difficult to seamlessly interface with the process control system because the RTA uses an off-the-shelf office-type personal computer (PC) that caused communication interference between the analysis software and the Operating System of the PC (OS).

U.S. Pat. Nos. 5,352,350, 6,458,262 B1, and 6,471,845 B1 disclose control of plating bath quality.

While predictive techniques have been used to control plating baths in semiconductor processing, most of these techniques combine offline or online measurements on the one hand with statistical techniques and process knowledge on the other. Whether heuristic or semi-empirical, these algorithms function in a discrete time fashion. However, the prediction error correction is seldom performed and the algorithm is set to operate only in a preset process window.

Current state-of-the-art analyzers used for process control are essentially on-line monitors/analyzers capable of measuring bath composition several times (generally 3 to 5 times) an hour. This relatively fast rate—compared to offline monitors—allows the implementation of “pseudo closed loop” or feedback control loop of any or all additives of interest. Due to the complex chemistry of plating baths, the required analysis time can hardly be shortened using state-of-the-art methods such as automated CVS or even AC-DC voltammetry. Real-time feedback control is never achieved in practice—only approximated at best, using predictive algorithms as additives concentrations are available several minutes apart, not to mention the need for some statistical calculations to provide meaningful data to the process control system, such as that disclosed in U.S. Pat. No. 6,471,845B1, entitled “Method of Controlling Chemical Bath Composition in a Manufacturing Environment”, which is incorporated by reference.

Therefore, those skilled in the art will also recognize that there is a need for a way to retrofit virtually any “real-time” analyzer or near real-time analyzers, to the existing process control system, be it the process tool or a chemical delivery system, as well as any other monitored locations using a state-of-the-art industrial automation solution. There is also a need to decrease and to even possibly eliminate human intervention. There is also a need to allow the possibility to perform real-time feedback process control on bath concentrations. Finally, there is a need for an industrial network readily usable for any existing or future connectivity and expandability needs.

In light of the problems associated with electroplating bath solutions having undesirable qualities, a need exists for improvement in the management of electroplating bath solutions.

### SUMMARY

The present invention provides a system for monitoring, dosing, and distribution of a chemical composition in a material treatment process, wherein the chemical composition contains at least one additive for maintaining the quality of the chemical treatment process. The system comprises at least one chemical containing unit configured to contain the chemical composition for the chemical treatment process a dosing unit fluidly communicating with at least one chemical containing unit configured to receive the chemical composition therefrom and to add a selected dose of at least one additive to the chemical composition therein, an online monitor configured to monitor a property of the chemical composition at the dosing unit and to transmit a signal corresponding to the monitored property, and a controller programmed and configured to receive the signal from the online monitor and send a signal to the dosing system to add the selected dose to the chemical composition therein in response to the monitored property.

The invention also provides a method for monitoring, dosing, and distribution of a chemical composition in a chemical treatment process, wherein the chemical composition contains at least one additive for maintaining the quality of the chemical treatment process. The method comprises the following steps. A flow of the chemical composition is allowed from at least one chemical containing unit to a dosing unit. A property of the chemical composition is monitored with an online monitor at a location intermediate to the chemical containing unit and the dosing unit or at the dosing unit. A signal associated with the monitored property is sent by the online monitor to a controller. The controller determines whether at least one additive should be added to the chemical composition at the dosing unit based upon the monitored property, thereby resulting in a decision to add or not a selected amount of at least one additive to the chemical composition at the dosing unit based upon the signal from the online monitor. A signal associated with the decision is sent from the controller to the dosing unit. The selected amount of at least one additive is allowed to be added or not be added to the chemical composition at the dosing unit in response to the signal associated with the decision. Thresholds can also be set which determine whether the additive is added, if, for example, the actual chemical concentration is still within acceptable concentration ranges, but near the low end of the range. The chemical composition is allowed to flow from the dosing unit to the chemical containing unit.

Additionally, the invention provides a method for controlling concentration of a chemical in a liquid bath during manufacturing, that includes predetermining a range of acceptable concentrations for one or more chemicals used in the bath during manufacturing, using an analyzer to measure concentration of one or more chemicals in the bath, where the measured concentration comprises a first data signal having a proprietary or non-standard communication protocol, transmitting the first data signal that corresponds to the measured concentration from the analyzer to a first computer, causing the first computer to transmit a second data signal corresponding to the first data signal to a second computer, the second signal having a proprietary communication protocol, and using hardware and/or software to cause the second computer to translate the second data signal having a proprietary communication protocol to a third data signal having an open or standard communication protocol. In a further embodiment of this invention, the controller comprises a processor programmed with an algorithm.

One embodiment of the invention is a system for controlling concentration of a chemical in a liquid bath during manufacturing, that includes: a predetermined range of acceptable concentration for at least one chemical used in the bath during manufacturing an analyzer for measuring a concentration of at least one chemical in the bath, where the analyzer provides a first data signal comprising measured concentration data, the data signal having a proprietary communication protocol, a first computer interfaced with the analyzer, where the first data signal is transmitted from the analyzer to the first computer and where the first computer transmits a second data signal corresponding to the first data signal to a second computer, the second signal having a proprietary communication protocol; hardware and/or software capable of causing the second computer to translate the second data signal having a proprietary communication protocol to a third data signal having an open communication protocol, and, transmitting the third data signal to a controller which controls the concentration of at least one chemical in the bath.

Another embodiment is a system for retrofitting a real-time analyzer (RTA) computer and a controller to improve real time control of chemical solutions used for material treatment process, that includes at least one networked real-time analyzer (RTA) computer, wherein the analyzer provides a first signal for transmitting data, the signal having a non-standard or proprietary communication protocol, a second computer for receiving the signal from the RTA, wherein the second computer converts the signal to a third signal having an open communication protocol, transmitting the third data signal to a controller that is capable of controlling the concentrations of the at least one chemical in the bath, and, an open communication protocol such as industrial Ethernet (802.3, 802.11i etc.), a TCP/IP protocol, a NetDDE, an OPC, or a combination of the foregoing and wherein the controller and the analyzer are interfaced using the communication protocol.

Yet another embodiment of the invention provides a method of maintaining a predetermined concentration of one or more chemicals in a liquid bath during manufacturing that includes predetermining an acceptable concentration range for at least one chemical used in the bath during manufacturing, predicting a depletion in concentration of at least one chemical that will occur during manufacturing using a controller to implement dosing of at least one chemical into the bath to correct the predicted depletion in concentration amounts to maintain the specified acceptable concentration range, using a real time analyzer to obtain a real time concentration of at least one chemical in the bath, transmitting the actual measured real time concentration to a controller, using a controller to compare the real time concentration with the predicted concentration range and determining any deviation between the concentrations, using the controller to determine and/or implement dosing amounts to be added to the bath to maintain the specified acceptable concentration range, comparing the real time concentration with the target concentration and determining any difference between the concentrations, and, using the controller to maintain the acceptable concentration range.

### BRIEF DESCRIPTION OF THE DRAWINGS

For a further understanding of the nature and objects of the present invention, reference should be made to the following detailed description, taken in conjunction with the accompanying drawings, in which like elements are given the same or analogous reference numbers and wherein:

FIG. 1 is a schematic of the inventive system including an optional chemical dispensing unit;

FIG. 2 is a schematic of an embodiment of the invention also including a dosing unit reservoir, a dosage element, a fresh chemical composition supply tank, a fluid bleed tank, a conduit allowing the chemical composition to bypass the chemical containing unit, and a conduit/valve arrangement allowing the chemical composition to bypass the dosing unit reservoir, dosage element, fresh chemical composition supply tank, bleed tank, and optional chemical dispensing unit;

FIG. 3 is a schematic of another embodiment of the invention also including multiple chemical containing units, a dosing unit reservoir, a dosage element, a fresh chemical composition supply tank, a bleed tank, a conduit allowing the chemical composition to bypass the chemical containing units, a conduit/valve arrangement allowing the chemical composition to bypass the dosing unit reservoir, dosage element, fresh chemical composition supply tank, bleed tank, and optional chemical dispensing unit, a manifold and reservoir downstream of the chemical containing units, and a second online monitor;

FIG. 4 is a schematic another embodiment of the invention also including multiple chemical containing units, a dosing unit reservoir, a dosage element, a fresh chemical composition supply tank, a bleed tank, a conduit allowing the chemical composition to bypass the chemical containing units, and a conduit/valve arrangement allowing the chemical composition to bypass the dosing unit reservoir, dosage element, fresh chemical composition supply tank, bleed tank, and optional chemical dispensing unit;

FIG. 5 is a schematic of a preferred configuration of the dosage element;

FIG. 6 is a schematic of a preferred configuration of the optional chemical dispensing unit;

FIG. 7 is a schematic illustrating existing process control systems and existing PC capabilities;

FIG. 8 is a schematic of a filter based virtual sensor for bath monitoring and control;

FIG. 9 is a schematic overview of the control method and system of this invention shown in a control block diagram;

FIG. 10 is a schematic illustrating an embodiment of the invention showing the main flow chart of program functions;

FIG. 11 is related to FIG. 10 and is a schematic illustrating the program functions of real time analysis;

FIG. 12 is related to FIG. 10 and is a schematic illustrating the program functions for requesting analysis;

FIG. 13 is related to FIG. 10 and is a schematic illustrating the program functions for additives concentration processing;

FIG. 14 is related to FIG. 10 and is a schematic illustrating the program functions for concentration monitoring;

FIG. 15 is related to FIG. 10 and is a schematic illustrating the program functions for additives dosing;

FIG. 16 is a schematic showing the interface of an RTA and industrial PC, utilizing the ISO OSI seven-layer communication model; and

FIG. 17 is a schematic further illustrating the parameters and tuning of an embodiment of a virtual sensor of this invention.

### DESCRIPTION OF PREFERRED EMBODIMENTS

The inventive system allows a chemical composition associated with a material treatment process to be monitored, dosed, and distributed to a chemical containing unit in which the dosing occurs at a location other than at the chemical containing unit. This lessens the risk of operator error and contamination because the frequency of changing the additive bottles at the chemical containing unit is decreased. For example, an incorrect bottle exchange will result in contamination.

When there is a plurality of chemical containing units, the risk of operator error and contamination is multiplied. Many chemical treatment facilities, such as semiconductor fabs or foundries utilize more than one wafer-plating tool in production. Since each tool is controlled separately, inconsistencies in the bath solution between tools can develop which can ultimately result in different properties of the copper interconnect deposit for different tools. Thus, the invention is especially advantageous with use with several chemical containing units.

Also, when a controller is integrated with online monitoring, the risk of operator error is lessened because of the relative lack of transliteration errors made by an operator in between analysis of offline data and decisions to add the additives. Thus, in the invention, process engineer intervention is not needed to correct the bath concentrations.

The controller is analyzer-independent and will not need any modifications if the bath management system were to be used in conjunction with different instrumentation systems. It only involves sequential discrete control thereby facilitating the control scheme and reducing the implementation cost.

In many prior art system, chemical consumption process model based on offline or online measurements is utilized to achieve bath control. However, the equations in these types of models are presumably only valid in the vicinity of a known operating regime and have limited ability to accommodate process upsets or disturbances. In contrast, the inventive controller does not rely upon a model, but instead makes additive decisions based upon real time analysis data.

This invention is especially advantageous when implemented in management of electroplating bath solutions wafer-plating tools in semiconductor fabs or foundries.

With reference to the Figures and Tables I-III, the inventive system and method and preferred embodiments thereof are illustrated.

TABLE I
First legend to reference characters in the figures

| | |
|---|---|
| 10 | inventive system |
| 5 | chemical containing unit |
| 11 | optional chemical delivery unit |
| 20 | piping from dosing unit to chemical delivery unit |
| 25 | Conduit |
| 40 | Conduit |
| 41 | communication link from controller to online monitor |
| 42 | communication link from controller to dosing unit |
| 45 | dosing unit |
| 55 | online monitor |
| 65 | Controller |
| 100 | preferred embodiment of the inventive system |
| 110 | chemical delivery unit |
| 112 | chemical delivery element |
| 120a | pressure-feed vessel 1 |
| 120b | pressure-feed vessel 2 |
| 130 | blanket gas system |
| 140 | Conduit |
| 150 | Bypass |
| 151 | Valve |
| 152 | Valve |
| 160 | day tank |
| 200 | Controller |
| 210 | communication link from controller to blending tank unit |
| 220 | communication link from controller to dosing unit |
| 230 | communication link from controller to optional chemical delivery |
| | unit |
| 240 | communication link from controller to monitor 1 |
| 245 | communication link from controller to monitor 2 |
| 250 | communication link from controller to chemical containing unit |
| 250a | communication link from controller to chemical containing unit |
| 250b | communication link from controller to chemical containing unit |
| 250c | communication link from controller to chemical containing unit |
| 250d | communication link from controller to chemical containing unit |
| 251 | communication link from controller to valve |
| 252 | communication link from controller to valve |
| 260 | communication link from controller solvent supply tank |
| 270 | communication link from controller to bleed tank |

TABLE II
Second legend to reference characters in the figures

| | |
|---|---|
| 300 | chemical containing element |
| 300a | chemical containing element 1 |
| 300b | chemical containing element 2 |
| 300c | chemical containing element 3 |
| 300d | chemical containing element 4 |
| 320 | inlet to chemical containing element |
| 320a | inlet to chemical containing element 1 |
| 320b | inlet to chemical containing element 2 |
| 320c | inlet to chemical containing element 3 |
| 320d | inlet to chemical containing element 4 |
| 330 | outlet from chemical containing element |
| 330a | outlet from chemical containing element 1 |
| 330b | outlet from chemical containing element 2 |
| 330c | outlet from chemical containing element 3 |
| 330d | outlet from chemical containing element 4 |
| 340 | chemical containing element outlet manifold |
| 350 | chemical containing element outlet reservoir |
| 360 | Pump |
| 400 | dosing unit reservoir |
| 415 | dosing unit circulation pump |
| 420 | piping from dosing unit to optional chemical delivery |
| | unit |
| 425 | piping from reservoir to dosage element |
| 426 | Conduit |
| 427a | Conduit |
| 427b | Conduit |
| 427c | Conduit |
| 427d | Conduit |
| 427e | Conduit |
| 428a | three way valve |
| 428b | three way valve |
| 428c | three way valve |
| 428d | three way valve |
| 429a | Conduit |
| 429b | Conduit |
| 429c | Conduit |
| 429d | Conduit |
| 430 | piping from dosage element to reservoir |
| 431 | Conduit |
| 435 | piping from conduit to fluid bleed tank |
| 437 | Valve |
| 440 | Pump |

TABLE III
Third legend to reference characters in the figures

| | |
|---|---|
| 450 | dosage element |
| 460 | piping from optional chemical delivery unit to chemical |
| | containing unit |
| 461 | piping for parallel loop |
| 471 | Valve |
| 472 | dosing pump |
| 473 | Valve |
| 474 | Flowmeter |
| 480a | chemical additive container |
| 480b | chemical additive container |
| 480c | chemical additive container |
| 480d | chemical additive container |
| 600 | fluid bleed tank |
| 610 | fluid bleed tank outlet |
| 700 | fresh chemical composition supply tank |
| 701 | piping from fresh chemical composition supply tank to dosing unit |
| 800 | Monitor |
| 810 | piping from dosing unit 400 to monitor 800 |
| 820 | piping from monitor |
| 850 | Monitor |
| 860 | piping to monitor |
| 870 | piping from monitor |

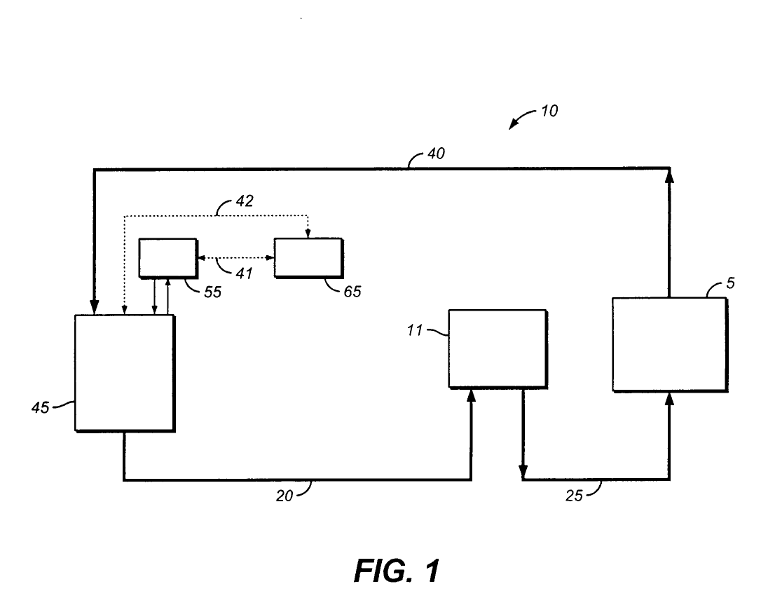

As illustrated in FIG. 1, the inventive system includes a chemical containing unit 5 through which the chemical composition flows. The chemical composition contains certain chemical constituents, such as additives of the chemical composition which are consumed and/or degraded in a material treatment process. The chemical containing unit 5 is operatively associated with the material treatment process which occurs either adjacently to chemical containing unit 5 or remotely therefrom. For example, when the chemical containing unit 5 may be located remotely from the material treatment process, it can be part of a larger chemical distribution system that incorporates bulk chemical distribution equipment. Preferably, the chemical containing unit is an electroplating bath reservoir associated with one or more electroplating (ECD) tools in the clean room of a fab and the dosing unit is located in a chemical room located in the sub-fab. More preferably, the electroplating tools are used to electroplate semiconductor wafers with copper. Also preferably, the chemical containing unit is an electrolyte reservoir for supplying an electrochemical planarization (ECP) electrode system in the clean room of a fab and the dosing unit is located in a chemical room located in the sub-fab. Also preferably, the chemical containing unit is a chemical reservoir for supplying a chemical mechanical planarization (CMP) system in the clean room of a fab and the dosing unit is located in a chemical room located in the sub-fab.

The chemical composition flows via conduit 40 to dosing unit 45. At dosing unit 45, the online monitor 55 monitors a property of the chemical composition. Preferably, the monitored property is a concentration of one or more chemical constituents of the chemical composition. More preferably, it is the concentration of one or more additives or of degradation products resultant from a chemical treatment process associated with the chemical composition operatively associated with the chemical containing unit 5.

The online monitor 55 may be any online monitor known to those ordinary skilled in the art suitable for use in the invention. A preferred online monitor is a “real-time analyzer”, RTA, available from Technic, Inc, Cranston, in Rhode Island, U.S.A. Another monitor suitable for use in the invention is the ECI system available from ATMI, Danbury, Conn., U.S.A.

The online monitor 55 sends a signal associated with the monitored property to the controller 65 via communication link 41. Based upon this signal, the controller 65 determines whether or not to add one or more additives to the chemical composition at the dosing unit 45, thereby resulting in a decision. The controller 65 sends a signal associated with the decision to dosing unit 45 via communication link 42. Based upon this signal, one or more additives are added or not to the chemical composition at the dosing unit 45. Preferably, the dosing unit 45 includes a mixing element so that the chemical composition and any additives are well mixed.

Preferred examples of additives include brighteners, suppressors, and levelers.

The chemical composition flows from the dosing unit 45 via conduit 20 to the optional chemical delivery unit 11. Optional chemical delivery unit 11 delivers the chemical composition to the chemical containing unit 5 via optional conduit 25. As the chemical delivery unit 11 and conduit 25 are optional, the inventive system 10 may be configured such that the chemical composition flows directly from the dosing unit 45 to the chemical containing unit via conduit 20.

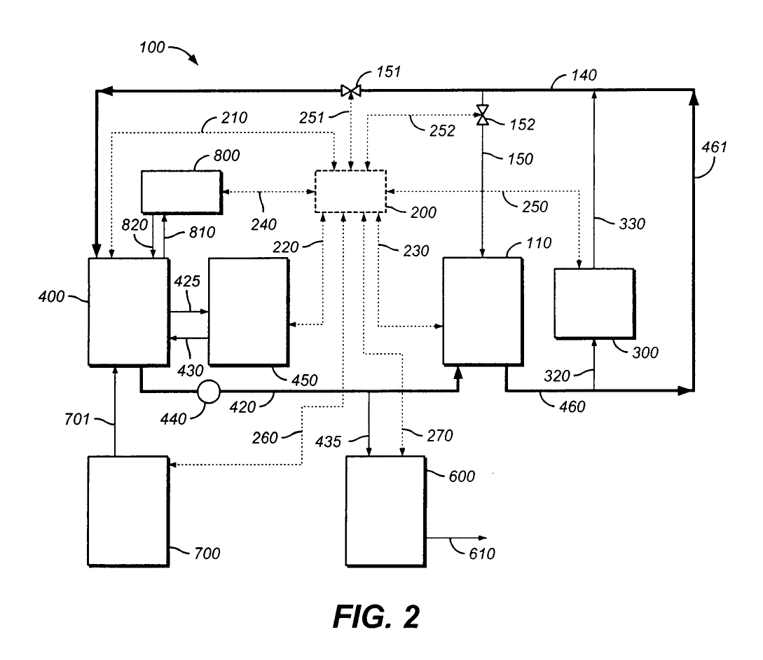

As best shown in FIG. 2, a preferred embodiment 100 of the inventive system includes a chemical containing unit 300 through which the chemical composition may flow either batch-wise or continuously. The chemical composition contains certain chemical constituents, such as additives of the chemical composition which are consumed and/or degraded in a material treatment process. The chemical containing unit 300 is operatively associated with the material treatment process which occurs either adjacently to chemical containing unit 300 or remotely therefrom. For example, when the chemical containing unit 300 may be located remotely from the material treatment process, it can be part of a larger chemical distribution system that incorporates bulk chemical distribution equipment.

Preferably, the chemical containing unit 300 includes inlet and outlet valves and a level sensor. When the level sensor senses a selected high level, the inlet and outlet valves close thereby preventing the flow of the chemical composition thereinto. When desired, an optional conduit 461 may be provided through which the chemical composition bypasses the chemical containing unit 300. Thus, the chemical composition may continue to flow through the system 100 when the inlet and outlet valves of chemical containing unit 300 are closed. This bypass flow may be implemented via signals sent between chemical containing unit 300 and controller 200 via communication link 250. This helps continue mixing of the chemical composition as well as maintaining a steady flow of it through the system, especially if the tool is disconnected.

The chemical composition flows from the chemical containing unit 300 via conduit 330 to conduit 140, and thenceforth past valve 151 and bypass conduit 150 to dosing unit reservoir 400. Dosing unit reservoir 400 and dosage element 450 together comprise a dosing unit analogous to dosing unit 45. At least a portion of the chemical composition is routed via piping 810 from the dosing unit reservoir 400 to online monitor 800, at which a property of the chemical composition is monitored. Preferably, the monitored property is a concentration of one or more chemical constituents of the chemical composition. More preferably, it is the concentration of one or more additives or of degradation products resultant from a material treatment process associated with the chemical composition operatively associated with the chemical containing unit 300.

The online monitor 800 sends a signal associated with the monitored property to controller 200. Based upon the signal, the controller 200 determines whether or not to add one or more additives at dosing unit reservoir 400 to the chemical composition, thereby resulting in a decision. A signal associated with this decision is sent from controller 800 via communication link 220 to the dosage element 450. In response to this signal, the dosage element 450 will or will not add one or more additives to the flow of chemical composition through piping 425 to dosage element 450. When additives are added to the chemical composition, the chemical composition with the added additives flows back to dosing unit reservoir 400 via piping 430. Preferably, the dosing unit 450 includes a mixing element so that the chemical composition and any additives are well mixed.

Also based upon the signal from online monitor 800, the controller 200 determines whether or not to supply fresh chemical composition via piping 701 from fresh chemical composition supply tank 700 to dosing unit reservoir 400. Based upon a signal from controller 200 via communication link 260, an outlet valve of the fresh chemical composition supply tank 700 opens and a suitable delivery means allows the fresh chemical composition to flow therefrom to dosing unit 400.

The fresh chemical composition is understood to be those portions of the chemical composition, excluding any additives added to the chemical composition by dosing unit reservoir 400 and any degradation products formed as a result of the material treatment process. When the invention is preferably applied to management of electroplating baths for wafer plating tools at a semiconductor fab or foundry, the fresh chemical composition is also referred to as fresh inorganic solution. In this instance, the fresh inorganic solution primarily contains CuSO₄, H₂SO₄, and a source of Cl⁻.

Also based upon the signal from online monitor 800, the controller 200 determines whether or not to bleed chemical composition at fluid bleed tank 600 via outlet 610. Based upon a signal from controller 200 via communication link 270, chemical composition is bled from conduit 420 via a valve associated with piping 435. Preferably, the fluid bleed tank 600 includes a level sensor. When the level sensor senses a selected high level, an outlet valve of the fluid bleed tank 600 opens and chemical solution exits, therefrom via outlet 610. In such a scenario, the valve and level sensor may optionally be associated with controller 200 and decisions to allow the chemical composition to exit from outlet 610 are made by the controller 200.

The fresh chemical composition at fresh chemical composition tank 700 may alternatively include one or more of the additives added to the chemical composition at dosing unit 400 or degradation products from the material treatment process. In that case, it contains only minimal concentrations of them. For example, spent chemical composition bled from fluid bleed tank 600 may be regenerated and then stored in fresh chemical composition supply tank 700.

With the aid of pump 440, the chemical composition flows from dosing unit reservoir 400 via conduit 420 to optional chemical delivery unit 110. Chemical delivery unit 110 includes a means suitable to deliver the chemical composition therefrom to chemical containing unit via optional conduit 460. The chemical composition then flows into the chemical containing unit 300 via inlet 320. It is understood that the system 100 may be configured such that the chemical composition flows directly from the dosing unit reservoir 400 to inlet 320 of chemical containing unit via conduit 420.

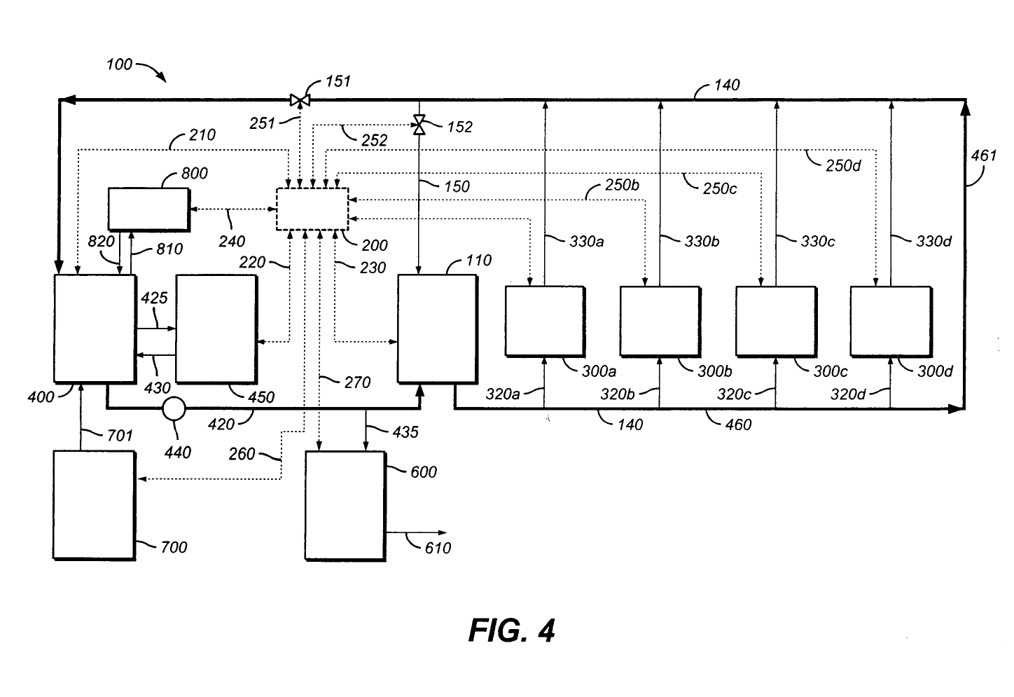

As best shown in FIG. 4, this embodiment 100 of the inventive system includes all the components illustrated in FIG. 2, except that four chemical containing units 300a-d and associated inlets 320a-d and outlets 330a-d along with associated communication links 250a-d are present. The benefits of implementing the invention with a plurality of chemical containing units is that such a centralized system helps to consistently maintain a chemical compositions having substantially the same properties at each of the chemical containing units even if they are running at different production capacities. In addition, one online monitor may be used for monitoring the bath composition for many tools, thereby reducing cost of ownership. Also, since the monitor and dosing unit is advantageously located in a separate location from the chemical containing units, chemical exchange and chemical handling are not performed in the location in which the material treatment process occurs. If the invention is implemented in management of electroplating bath solutions of wafer-plating tools in semiconductor fabs or foundries, the dosing unit and monitor may be located in the sub-fab room or chemical room, rather than the cleanroom area of the fab. Thus, less handling is done in the cleanroom thereby lessening the risk of contamination. In addition, there is a potentially smaller footprint in the cleanroom.

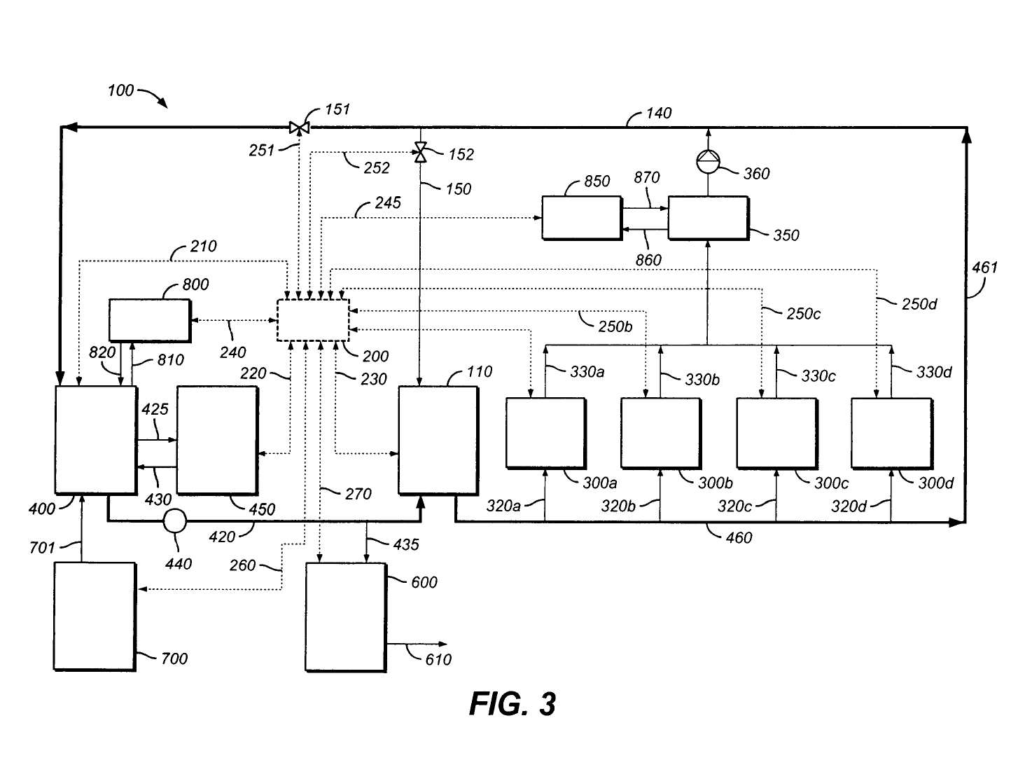

As best shown in FIG. 3, another preferred embodiment 100 of the system includes the same components illustrated in FIG. 2, except that four chemical containing units 300a-d, associated with inlets 320a-d, outlets 330a-d, and communication links 250a-d are used, and it further includes a second monitor 850 associated with a communication link 245 between the monitor 850 and controller 200, a chemical containing unit outlet reservoir 350, and a pump 360. Operation of this embodiment allows control over the quality of multiple chemical containing units, such as four chemical containing units 300a-d, in a centralized scheme.

In this embodiment, monitor 850 monitors a property of the chemical composition at reservoir 350 via piping 860 and returns it to reservoir 350 via piping 870. This embodiment is especially advantageous when the monitor 800, dosing unit reservoir 400 and dosage element 450 are located far from the chemical containing units 300a-d. For example, when the invention is implemented for management of electroplating baths of wafer-plating tools, the chemical containing units 300a-d (in this case wafer-plating tools) are located in the fab and the dosing unit reservoir 400, dosage element 450, and monitor 800 are located in the sub-fab room. Whether or not the invention is applied to management of these baths, the following situation occurs. For a given plug of chemical composition flow, monitoring of a property by monitor 850 and monitor 800 may result in different measurements due to the lag time of the plug's flow between reservoir 350 and dosing unit reservoir 400. In order to help compensate for this difference, monitor 850 and monitor 800 together provide signals to controller 200 regarding the monitored property via communication links 245 and 240. Based upon these signals, the controller 200 will implement the best decision regarding the addition of one or more additives to the chemical composition. Also, if monitor 800 detects potential contamination of the chemical composition, selected valves isolating the chemical composition in the chemical treatment units and associated piping and the dosing unit may be shut down so as to not contaminate the whole system.

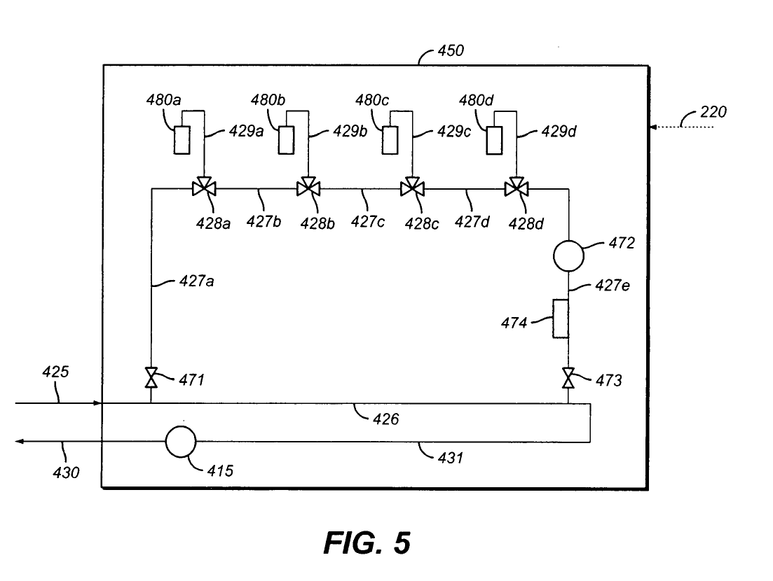

A preferred embodiment of the dosage element 450 is best illustrated in FIG. 5. During a period in which the additives are not being added to the chemical composition, flow of the composition to dosage element 450 via piping 425 does not ordinarily occur (by action of closed valves not shown). When the controller 200 sends a signal to the dosage element to begin dosing the additives to the composition, pump 415 is activated and the composition flows to dosage element 450 via piping 425 into conduit 426. Addition to the chemical composition of additive from additive container 480a is described below.

At the moment that dosage element 450 receives a signal from the controller 200 to begin dosing the additive from container 480a, a flushing cycle is initiated. In the flushing cycle, valve 471 is opened. This operation lasts for a specified time in order to flush the contents of the line from valve 471 to valve 473. After the flush has been completed, the pump is deactivated, and valves 473, 471 are closed. The dosing element 450 receives another signal from the controller 200 to begin dosing the additive from container 480a, the three way valve 428a is opened, the pump 472 is activated, and valve 473 is opened. At this point, the upstream portion of three-way valve 428a is closed while the other two portions are open. Also the in-line portions of valves 428b, 428c and 428d are opened while the remaining portions of these valves adjacent conduits 429b, 429c and 429d remain closed. By action of pump 472, the additive from container 480a flows through conduit 429a and past conduit 427b, open three-way valve 428b, conduit 427c, open three-way valve 428c, conduit 427d, open three-way valve 428d, and into conduit 427e past pump 472. The amount of the additive is preferably controlled by one of two methods. The flow rate at flow meter 474 is monitored during the flushing cycle and then integrated over time, thereby yielding a relationship between the volume of flow past the pump 472 and the time during which the pump 472 is activated. Based upon this time/flow relationship, the amount of time which pump 472 is activated during the dosing determines the volume of additive added.

When the selected amount of additive from container 480a is determined to be introduced into conduit 427b or downstream thereof, the upstream and downstream portions of three-way valve 428a adjacent conduits 427a and 427b are opened and the remaining portion adjacent conduit 429a is closed. Substantially simultaneously, valve 471 is opened and by action of pumps 472 and 415, the chemical composition flows into conduit 427a via conduit 426 and piping 425. The in-line portions of three-way valves 428b, 428c and 428d are open, thereby allowing the chemical composition to flow through conduits 427b, 427c, 427d, 427e, and valve 473. The chemical composition then enters conduit 431 where it is directed to piping 430 via pump 415 and into the dosing unit reservoir 400. If the controller 200 indicates that no other additive should be dosed, once a selected amount of chemical composition has flowed through this loop of conduits parallel conduit 426, valves 471 and 473 are closed and pumps 472 and 415 shut down. At this point, flow of the chemical composition between dosing unit reservoir 400 and dosage element 450 is interrupted.

Those skilled in the art will understand that addition of any other of the additives from containers 480b, 480c or 480d may be achieved in the same manner with the addition and flushing sequences as described above.

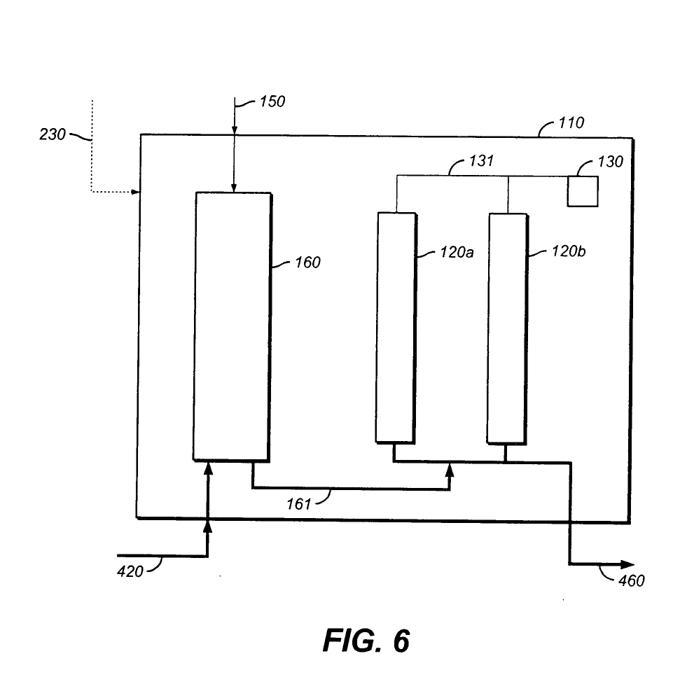

A preferred embodiment of chemical delivery unit 110 is best illustrated in FIG. 6. Chemical composition flows into day tank 160 via either conduits 150 or 420. The chemical composition flows out of day tank 160 through conduit 161 and into pressure-feed vessel 120a, while at the same time chemical composition flows out of pressure-feed vessels 120b and into conduit 460 by action of blanket gas system 130. Flow of chemical composition through conduit 161 and into or out of pressure feed vessels 120a and b is achieved by known arrangements of valving in order to avoid mixing the two flows. When a sensor in pressure-feed vessel 120a senses a high liquid level and a sensor in pressure-feed vessel 120b senses a low liquid level, flow of the chemical composition thereinto is interrupted, while at the same time, flow out of pressure-feed vessel 120b is also interrupted. At this point, the filling and emptying of the two pressure-feed vessels 120a, b is switched, i.e., pressure-feed vessel 120a is emptied by flow of chemical composition out of it and into conduit 460 by blanket gas system 130, while pressure-feed vessel 120b is being filled. Pressure sensors may be used to determine the beginning and end of a fill or empty cycle as described above instead of liquid level sensors.

The online monitor 800 may be any online monitor known to those ordinarily skilled in the art suitable for use in the invention. A preferred online monitor is a “real-time analyzer”, RTA, available from Technic, Inc, Cranston, in Rhode Island, U.S.A. Another monitor suitable for use in the invention is the ECI system available from ATMI, Danbury, Conn., U.S.A.

Optionally and in some situations, based upon a signal from online monitor 800, the controller 200 determines that the flow of chemical composition should bypass the dosing unit reservoir 400. This situation is especially desirable when the controller 200 determines that the one or more additives should be added to the chemical composition at dosing unit reservoir 400 by dosage element 450.

The above bypass is implemented in the following manner. Controller 200 determines whether or not to allow the flow of chemical composition to bypass the dosing unit reservoir 400. A signal based upon this determination is sent from controller 200 to valves 151 and 152 via communication links 251 and 252 to allow the valves to remain open, remain closed, open from a closed position, or close from an open position. When chemical composition is flowing from chemical containing unit 300 to dosing unit reservoir 400 via outlet 330 and conduit 140, a decision by the controller 200 to allow the chemical composition to bypass these components results in a signal from controller 200 to close valve 151 and open valve 152. The chemical composition then leaves conduit 140, enters conduit 150 and flows to chemical delivery unit 110. In this instance, pump 440 is actuated such that the flow of chemical composition from dosing unit reservoir 400 to the optional chemical delivery unit is interrupted. Actuation of the pump 440 is preferably controlled by the controller 200 at the same time the bypass is implemented. Otherwise, the pump 440 could be damaged.

As described above, the flow of chemical composition may optionally be allowed to bypass the dosing unit reservoir 400 because the controller 200 determines that one or more additives should be added to the chemical composition. In this instance and contemporaneously with the bypass of the flow of chemical composition via conduit 150 as described above, a flow of chemical composition to and from the dosing unit reservoir 400 and dosage element 450 via piping 425 and 430 occurs. The one or more additives are then added to the chemical composition by dosage element 450 as described above. When addition to the chemical composition of the one or more additives is completed and the combined chemical composition and additives enter the dosing unit reservoir 400, controller 200 sends signals to valve 151 to open and valve 152 to close, thereby allowing the chemical composition to flow from chemical containing unit 300 to dosing unit reservoir 400.

Preferably, when one or more additives are being added to the chemical composition by dosage element 450, an otherwise interrupted flow of chemical composition from/to the dosing unit reservoir 400/dosage element 450 via piping 425 and 430 is allowed to commence. This may occur by actuation of one or more valves to close and operation of one or more pumps to be interrupted. At the same time, the chemical composition from chemical containing unit 300 bypasses dosing unit reservoir 400 as described above.

Also, preferably, when addition to the chemical composition of the one or more additives is completed, the flow of chemical composition from/to the dosing unit reservoir 400/dosage element 450 via piping 425 and 430 ceases. At the same time, controller 200 sends signals to valve 151 to open and valve 152 to close, thereby allowing the chemical composition to flow from chemical containing unit 300 to dosing unit reservoir 400.

It should be understood that valves 151,152 could be electric solenoid or pneumatic valves. In the case of electric solenoid valves actuation of valves 151, 152 is performed as described above, i.e., by the sending of a signal from controller 200. However, it should be also be understood that in the case of pneumatic valves, they need not be actuated directly by controller 200 via signals along communication links 251, 252. For example, the controller may send a signal to a source of compressed air which then pressurizes a pneumatic line which ultimately opens or closes a pneumatic valve. The source of compressed may be located adjacent the controller 200, at the valve 151, 152, or any point therebetween. For that matter, any pneumatic valve or pump may be controlled by controller 200 in this manner.

In these above preferred situations, two modes of chemical composition flow are described. When no addition of one or more additives to the chemical composition occurs, a main recirculation mode is present and the chemical composition is allowed to flow from the chemical containing unit 300 to the dosing unit reservoir 400 and thenceforth to optional chemical delivery unit 110 via conduit 420 and optional conduit 46 to chemical containing unit 300. This flow is referred to as loop C.

When addition of one or more additives to the chemical composition does occur, a dosing mode is present and the chemical composition flows from the chemical containing unit 300 through outlet 330, conduit 150 to chemical delivery unit 110 and thence forth to the chemical containing unit 300 via conduit 460 and inlet 320. This flow is referred to as loop A. In the same mode and at the same time, the chemical composition flows from/to the dosing unit reservoir 400/dosage element 450 via piping 425 and 430. This flow is referred to as loop B. The benefits of such a two-mode system are that solution recirculation feeding the chemical containing unit (or wafer-coating tool if the invention is implemented in management of electroplating bath solutions) can still be done while dosing inside the dosing unit is occurring. Thus continuous chemical delivery is achieved.

Predictive Corrective Controller:

While any one of the closed-loop control schemes known in the art may be used with controllers 65, 200, the inventors have invented a particularly advantageous control solution that overcomes the disadvantages presented by the prior art. The solution is a computer including but not limited to a programmable logic controller (PLC), a computerized control system, an Industrial PC, an personal computer, or the like, a combination of one or more of the foregoing, as well as any other computers or computing software and/or hardware known to one-skilled in the art for such purposes. In an embodiment, the computer is suitably programmed with a closed loop feedback dosing algorithm that utilizes actual process concentration data to correct for consumption of critical components in the chemical composition. It may also provide a predicted dosing volume in-between actual concentration data times, if the later are too large. It may also correct the dose when an actual value becomes available. It maintains the levels of one or more additives within a desired range by specifying amounts of additives and/or fresh chemical composition to be added to the chemical composition, as well as specifying when preset volumes and/or amounts of at least one chemical composition should be bled from the system.

The algorithm has several advantages. It is substantially independent of any changes in the kinetics of the chemical composition constituents, such as consumption rates, production mode, as well as independent of process tool specificities. It is also substantially independent of changes in the chemical composition's chemistry, such as variance of bulk chemical supplies, acidity, etc. Also, it is not dependent upon any one type of online monitor. Additionally, it is also scalable and thus able to support multiple chemical composition management configurations. Furthermore, the dosing frequency is tunable and dosing could take place even if actual process concentration data are not continuously available. In other words, addition of one or more additives may be made in between actual measurements of one or more properties by a monitor. Finally, the controller is robust enough to tolerate variable bath bleeding rates and typical process disturbances, such as addition of deionized water.

Due to the long sampling time of current online analyzers, the algorithm will predict concentrations at a desired frequency, also known as the dosing frequency. Each set of data either monitored or predicted is inspected by a diagnostic function to determine whether the operating mode of the system should be switched from the main recirculation mode to the dosing mode. If addition of one or more additives is determined by the algorithm as necessary, the controller will put the system in the dosing mode. As a result, the flow through loop C will be interrupted and flows through loops A and B commenced.

The algorithm will compute additive and fresh chemical composition amounts and/or volumes to bring all chemical composition concentrations of interest back to setpoint values. The calculated volumes are introduced into the system as described above. When the chemical composition and additives are deemed to be suitably mixed, the system is switched back from the dosing mode to the main recirculation mode and flow through loop C resumed.

In the absence of any control, the additive concentrations in the chemical composition will naturally deplete as a result of the ongoing material treatment process. If no action is taken, such concentrations will eventually cross below a threshold resulting in improper material treatment. In the case of electroplating bath solutions for wafer-coating, such phenomena as unacceptable metal deposition and formation of voids may occur.

For each additive, the dosing aims to re-establish target concentration in the solution. At time t, flows of the chemical composition through loops A and B commence. The concentration in the chemical containing unit will continue to decrease at rate r(t) due to the on-going material treatment process. The dosing is performed in the dosing unit by adding a volume V_{dosed}(t) of nominal additive at time t, which depends upon the chosen concentration units utilized by the real time monitor and the concentration of the chemical additives. Such a volume will make the system's additive concentration within a predetermined concentration range around the setpoint when the recirculation mode commences again at time t+Δt.

Various aspects of the predictive corrective algorithm are now described as follows.

TABLE IV
Nomenclature

| Component Symbol | | |
|---|---|---|
| Continuous | Discrete | |
| Time | Time | Description |
| V_{PT} | | Chemical containing unit volume |
| V_{BT} | | Dosing unit reservoir volume |
| V_{dosed}(t) | Vᵢ | Volume Dosed at time t or tᵢ |
| V_{bleed}(t) = α(t) · V_{BT} | αᵢ · V_{BT} | Volume bled at time t or tᵢ |
| C₀ | | Target concentration in chemical |
| | | containing unit |
| C | | Nominal concentration of chemical |
| | | additive |
| t | tᵢ | Time the dosing is started or i^{th} |
| | | dosing time |
| | θ_{RTA} | Monitor time delay |
| | ${f}_{s} = \frac{1}{{θ}_{RTA}}$ | Monitor sampling frequency |
| Δt = N · θ_{RTA}, | Δt = tᵢ₊₁ − tᵢ | Dosing period |
| N ∈ {1, 2, . . .} | | |
| ${f}_{d} = \frac{1}{Δt}$ | | Dosing frequency |
| C_{PT}(t) | C_{i,PT} | Concentration in chemical |
| | | containing unit at time t or tᵢ |
| C_{PT}(t + Δt) | C_{i+1,PT} | Concentration in chemical |
| | | containing unit at time t + |
| | | Δt or tᵢ₊₁ |
| C_{BT}(t) | C_{i,BT} | Concentration in dosing unit |
| | | reservoir at time t or tᵢ |
| C(t) | Cᵢ | Concentration in chemical |
| | | containing unit or dosing unit |
| | | reservoir at time t or tᵢ |
| $r ( t ) = \dot{C ( t )} = \frac{ⅆ C}{ⅆ t}$ | ${r}_{i} = {\dot{C}}_{i}$ | Chemical Consumption Rate at time t or tᵢ |
| $δ ( t ) = \ddot{C} ( t ) = \frac{{d}^{2} C}{{d t}^{2}}$ | ${δ}_{i} = {\ddot{C}}_{i}$ | Consumption Rate variation at time t or tᵢ |
| \hat{X}(t) | \hat{X}ᵢ | Estimated value of physical |
| | | quantity X (C, r, . . . etc) at |
| | | time t or tᵢ |

TABLE VI
Assumptions

| Assumption | | Continuous Time | Discrete Time |
|---|---|---|---|
| 1 | Perfect mixing | C_{PT}(t) = C_{BT}(t) = C(t) | C_{i,PT} = C_{i,BT} = Cᵢ |
| | during recirculation | | |
| 2 | Dosing reservoir is | C_{BT}(t) = C_{BT}(t + Δt) | C_{i,BT} = C_{i+ 1,BT} |
| | reaction free: | | |
| 3 | Constant Consumption Rate over θ_{RTA}: | $\begin{matrix}r ( t - Δt ) = r ( t - {θ}_{RTA} ) ∀ Δt ∈ [ 0 , {θ}_{RTA} ] \\ \begin{matrix}{C}_{PT} ( t ) = {\int }_{t - Δt}^{t} r ( t - Δt ) ⅆ x + {C}_{PT} ( t - Δt ) \\ = r ( t - {θ}_{RTA} ) Δt + {C}_{PT} ( t - Δt )\end{matrix}\end{matrix}$ | C_{i,PT} = rᵢ₋₁Δt + C_{i-1,PT} |
| 4 | Constant Consumption Rate variation over 2θ_{RTA}: | $\begin{matrix}δ ( t - Δt ) = δ ( t - 2 {θ}_{RTA} ) ∀ Δt ∈ [ 0 , {θ}_{RTA} ] \\ \begin{matrix}r ( t ) = {\int }_{t - Δt}^{t} δ ( t - Δt ) ⅆ x + r ( t - Δt ) \\ = 2 δ ( t - 2 {θ}_{RTA} ) Δt + r ( t - Δt )\end{matrix}\end{matrix}$ | rᵢ = δᵢ₋₁Δt + rᵢ₋₁ |

1. Dosing Algorithm for Single Component Case:

a) Continuous-Time Single Component Dosing (CTSC)

In one preferred embodiment, dosing is performed in the dosing unit reservoir 400 by adding a volume V_{dosed}(t) of nominal solution at time t. Such volume will depend upon the chosen concentration units utilized by the real time monitor and the concentration of the chemical additives, which enables the system's concentration to reach its target value at time t+Δt. This statement translates into the following equation:
(Mass in System at t+Δt)=(Mass in dosing unit reservoir at t)+(Mass added into dosing unit reservoir at t)+(Mass in chemical containing unit at t+Δt)−(Mass bled off the System at t) Eq. 1

$$\begin{matrix}{C}_{0} \cdot ( \begin{matrix}{V}_{BT} + {V}_{PT} + \\ {V}_{dosed} ( t )\end{matrix} ) = {C}_{BT} ( t ) \cdot {V}_{BT} + \overline{C} \cdot {V}_{dosed} ( t ) + \\ {C}_{PT} ( t + Δ t ) \cdot \\ {V}_{PT} - {V}_{bleed} ( t ) \cdot C ( t ) \\ = ( 1 - α ( t ) ) \cdot C ( t ) \cdot {V}_{BT} + \overline{C} \cdot {V}_{dosed} \\ ( t ) + C ( t + Δ t ) \cdot {V}_{PT}\end{matrix} \qquad Eq . 2$$

V_{dosed}(t) may be solved:

$${V}_{dosed} ( t ) = \frac{\begin{matrix}[ {C}_{0} - ( 1 - α ( t ) ) \cdot C ( t ) ] \cdot \\ {V}_{BT} + [ {C}_{0} - C ( t + Δ t ) ] \cdot {V}_{PT}\end{matrix}}{\overline{C} - {C}_{0}} \qquad Eq . 3$$

In the previous expression the concentrations at time t and at time t+Δt are unknown. It takes θ_{RtA} for the monitor to generate one set of results. Only C (t−θ_{RTA}) and the previous rate are available through the monitor 800 and/or direct computation respectively. Using assumption 3, one can estimate the concentration at time t:
Ĉ(t)=r(t−Δt)Δt+C(t−Δt) Eq. 4

Furthermore, the concentration at the end of the dosing period i.e. at t+Δt is not known. The consumption rate is not necessarily constant from one sampling period to the next. However, using assumption 4, one can estimate the consumption rate:
\hat{r}(t)=δ(t−Δt)Δt+r(t−Δt) Eq. 5

Thus, the estimated concentration at t+Δt using Eq. 4 and Eq. 5 is:

$$\begin{matrix}\hat{C} ( t + Δ t ) = \hat{ r } ( t ) Δ t + \hat{C} ( t ) \\ = [ δ ( t - Δ t ) Δ t + r ( t - Δ t ) ] \cdot Δ t + r ( t - Δ t ) Δ t + \\ C ( t - Δ t ) \\ = δ ( t - Δ t ) {( Δ t )}^{2} + 2 r ( t - Δ t ) Δ t + C ( t - Δ t )\end{matrix} \qquad Eq . 6$$

Eq. 3 may be rewritten by replacing the concentration at time t [C(t)] and consumption rate [\hat{r}(t)] by their estimated values in Eq. 4 and in Eq. 6 respectively to determine the volume, which depends upon the chosen concentration units utilized by the real time monitor and the concentration of the chemical additives, to be dosed into the system:

$${\hat{V}}_{dosed} ( t ) = \frac{\begin{matrix}\{ {C}_{0} - ( 1 - α ( t ) ) \cdot [ r ( t - Δ t ) + C ( t - Δ t ) ] \vert \} \cdot \\ {V}_{BT} + \\ \{ \begin{matrix}{C}_{ 0} - \vert δ ( t - Δ t ) {( Δ t )}^{2} + 2 r \\ ( t - Δ t ) Δ t + C ( t - Δ t ) \vert\end{matrix} \} \cdot {V}_{PT}\end{matrix}}{\overline{C} - {C}_{0}} \qquad Eq . 7$$

b) Discrete Time Single Component (DTSC) Dosing Algorithm:

In another preferred embodiment, the CTSC dosing algorithm utilizes a discrete time controller to control the chemical composition concentration. Like in the continuous case, the volume Vᵢ of nominal solution, which depends upon the chosen concentration units utilized by the real time monitor and the concentration of the chemical additives, at time tᵢ will make the system's concentration reach its target value when the blending tank unit 400 is interfaced at time tᵢ₊₁. This statement translates into the following equation:
(Mass in System at tᵢ₊₁)=(Mass in dosing unit reservoir at tᵢ)+(Mass added to g unit reservoir at tᵢ)+(Mass in chemical containing unit at tᵢ₊₁)−(Mass off the System at tᵢ) Eq. 1′
C₀·(V_{BT}+V_{PT}+Vᵢ)=Cᵢ·V_{BT}+\overline{C}·Vᵢ+Cᵢ₊₁·V_{PT}−αᵢ·V_{BT}·Cᵢ Eq. 2′

Vᵢ may be solved:

$${V}_{i} = \frac{[ {C}_{0} - ( 1 - {α}_{i} ) \cdot {C}_{i} ] \cdot {V}_{BT} + [ {C}_{0} - {C}_{i + 1} ] \cdot {V}_{PT}}{\overline{C} - {C}_{0}} \qquad Eq . 3 '$$

The concentrations Cᵢ and Cᵢ₊₁ are unknown. Using assumption 3, one can estimate the concentration at time tᵢ:

$${\hat{C}}_{i} = {r}_{i - 1} Δ t + {C}_{i - 1} = {\sum }_{k = 0}^{i - 1} {r}_{k} Δ t + {C}_{0} \qquad Eq . 4 '$$

Using assumption 4, one can estimate the consumption rate:

$${\hat{r}}_{i} = {δ}_{i - 1} Δ t + {r}_{i - 1} = {\sum }_{k = 0}^{i - 1} {δ}_{k} Δ t + {r}_{0} \qquad Eq . 5 '$$

The estimated concentration at tᵢ₊₁ using Eq. 4′ and Eq. 5′ is:

$${\hat{C}}_{i + 1} = ( {\sum }_{k = 0}^{i - 1} {δ}_{k} Δ t + {r}_{0} ) Δ t + {\sum }_{k = 0}^{i - 1} {r}_{k} Δ t + {C}_{0} \qquad Eq . 6 '$$

Eq. 3′ may be rewritten using Eq. 4′ and Eq. 6′ to determine the volume, which depends upon the chosen concentration units utilized by the real time monitor and the concentration of the chemical additives, to be dosed into the system:

$${\hat{V}}_{i} = \frac{\begin{matrix}( {α}_{i} \cdot {C}_{0} - ( 1 - {α}_{i} ) \cdot {\sum }_{k = 0}^{i - 1} {r}_{k} Δ t ) {V}_{BT} + \\ ( {\sum }_{k = 0}^{i - 1} {δ}_{k} Δ t + {r}_{0} + {\sum }_{k = 0}^{i - 1} {r}_{k } ) Δ t \cdot {V}_{PT}\end{matrix}}{{C}_{0} - \overline{C}} \qquad Eq . 7 '$$

2. Dosing Algorithm for Multi-Component Case:

a) Discrete Time Multi-Component (DTMC) Dosing Algorithm:

In another preferred embodiment, multiple components are controlled using a discrete time algorithm in the controller 200. Like in the single component case, the volume V_{i,j} of Component j nominal solution at time tᵢ will make the system's j^{th} concentration reach its target value when the solution in the dosing unit reservoir 400 is circulated back to the chemical containing unit 300 at time tᵢ₊₁. This statement translates into the following equation:
(Mass of j^{th} Component in System at tᵢ₊₁)=(Mass of j^{th} Component in dosing unit reservoir at tᵢ)+(Mass of j^{th} Component added to dosing unit reservoir at tᵢ)+(Mass of j^{th} Component in chemical containing unit at tᵢ₊₁)−(Mass of j^{th} Component bled off the System at tᵢ) Eq. 1″

$${C}_{0 , j} \cdot ( \begin{matrix}{V}_{BT} + {V}_{PT} + \\ {\sum }_{k = 1}^{n} {V}_{i , k}\end{matrix} ) = \begin{matrix} {C}_{i , j} \cdot {V}_{BT} + {\overline{C}}_{j} \cdot {V}_{i , j} + \\ {C}_{i + 1 , j} \cdot j ∈ [ 1 , n ] \\ {V}_{PT} - {α}_{i} \cdot {V}_{BT} \cdot {C}_{i , j }\end{matrix} \\ Let {C}_{i} = [ \begin{matrix}{C}_{i , 1} \\ ⋯ \\ {C}_{i , j} \\ ⋯ \\ {C}_{i , n}\end{matrix} ] , \\ {V}_{i} = [ \begin{matrix}{V}_{i , 1} \\ ⋯ \\ {V}_{i , j} \\ ⋯ \\ {V}_{i , n}\end{matrix} ] and \\ A = [ \begin{matrix}( {C}_{0 , 1} - {\overline{C}}_{1} ) & {C}_{0 , 1} & ⋯ & ⋯ & {C}_{0 , 1} \\ ⋯ & ⋯ & ⋯ & ⋯ & ⋯ \\ {C}_{0 , j} & ⋯ & ( {C}_{0 , j} - {\overline{C}}_{j} ) & ⋯ & {C}_{0 , j} \\ ⋯ & ⋯ & ⋯ & ⋯ & ⋯ \\ {C}_{0 , n} & ⋯ & ⋯ & {C}_{0 , n} & ( {C}_{0 , n} - {\overline{C}}_{n} )\end{matrix} ] \qquad Eq . 2 ''$$

Using the above notations, Eq. 2″ becomes:
Vᵢ=A⁻¹·[((1−αᵢ)·C₁−C₀)·V_{BT}+(Cᵢ₊₁−C₀)·V_{PT}] Eq. 3″

In the previous expression the concentration vectors Cᵢ and Cᵢ₊₁ are unknown but can be estimated. If

$${r}_{i} = [ \begin{matrix}{r}_{i , 1} \\ ⋯ \\ {r}_{i , j} \\ ⋯ \\ {r}_{i , n}\end{matrix} ]$$

the rate vector, then the current (i.e. at time tᵢ) concentration vector:

$$\begin{matrix}{\hat{C}}_{i} = {r}_{i - 1} Δ t + {C}_{i - 1} \\ = {\sum }_{k = 0}^{i - 1} {r}_{k} Δ t + {C}_{0}\end{matrix} \qquad Eq . 4 ''$$

Like in the single component case, one can estimate the current consumption rate vector:

$$\begin{matrix}{\hat{r}}_{i} = {δ}_{i - 1} Δ t + {r}_{i - 1} \\ = {\sum }_{k = 0}^{i - 1} {δ}_{k} Δ t + {r}_{0}\end{matrix} \qquad Eq . 5 ''$$

The concentration vector may be estimated at tᵢ₊₁ using Eq. 4″ and Eq. 5″:

$${\hat{C}}_{i + 1} = ( {\sum }_{k = 0}^{i - 1} {δ}_{k} Δ t + {r}_{0} ) Δ t + {\sum }_{k = 0}^{i - 1} {r}_{k} Δ t + {C}_{0} \qquad Eq . 6 ''$$

Eq. 3″ may be rewritten using Eq. 4″ and Eq. 6″ to determine the volume, which depends upon the chosen concentration units utilized by the real time monitor and the concentration of the chemical additives, to be dosed into the system:

$$\begin{matrix}{\hat{V}}_{i} = {A}^{- 1} \cdot [ ( 1 - {α}_{i} ) \cdot {\sum }_{k = 0}^{i - 1} {r}_{k} Δ t - {α}_{i} \cdot {C}_{0} ) {V}_{BT} + \\ ( {\sum }_{k = 0}^{i - 1} {δ}_{k} Δ t + {r}_{0} + {\sum }_{k = 0}^{i - 1} {r}_{k} ) Δ t \cdot {V}_{PT} ]\end{matrix} \qquad Eq . 7 ''$$

b) Corrected Discrete Time Multi-Component (CDTMC) Dosing Algorithm:

In another preferred embodiment, a monitor is used to correct the DTMC dosing volume. The monitor utilized for concentration measurements can have a significant dead time θ_{RTA}. In other words:

$$\{ \begin{matrix}{C}_{measured } ( t ) = {C}_{actual} ( t - {θ}_{RTA} ) , & 0 \leq t < ∞ \\ {C}_{measured } ( t ) = 0 , & t < 0\end{matrix}$$

The monitor transfer function in the s-domain (frequency domain) is C_{measured}(s)=e^{-sθ}^{_{RTS}}·C_{actual}(s). However, this formulation is impractical as the actual concentration is being sampled. Therefore, a discrete time representation in the z-domain may be utilized: C_{i,measured}=z^{-θ}^{_{RTA}}·C_{i,actual} where z=e^{s·θ}^{_{RTA}} meaning that C_{i,j}^{measured}=z^{-θ}^{_{RTA}}·C_{i,j}^{actual}=C_{i-1,j}^{measured}.

Due to the monitor dead time, Cᵢ is available at tᵢ₊₁ and Cᵢ₊₁ at tᵢ₊₂. Therefore at tᵢ₊₂, one must correct the dosed volume calculated based on the reading at time tᵢ.

The error of the dosed volume of the j^{th} at tᵢ₋₂ is defined and corrected at tᵢ as follows:

$$\{ \begin{matrix}{ɛ}_{i , j} = {λ}_{j} \cdot ( {V}_{i - 2 , j} - {\hat{V}}_{i - 2 , j} ) + ( 1 - {λ}_{j} ) \cdot {ɛ}_{i - 1 , j} , i \geq 2 \\ {ɛ}_{1 , j} = {ɛ}_{0 , j} = 0\end{matrix} \qquad Eq . 8 ''$$

This exponentially weighted moving average error is either positive or negative. To demonstrate that the error ε_{i,j} is a weighted average of all the previous deviations of the estimated volume from the real dosed volume, recursively for ε_{i-k,j} is substituted recursively:

$$\{ \begin{matrix}{ɛ}_{i , j} = \begin{matrix}{λ}_{j} \cdot {\sum }_{k = 2}^{i} {( 1 - {λ}_{j} )}^{j - k} \cdot ( \begin{matrix}{V}_{k - 2 , j} - \\ {\hat{V}}_{k - 2 , j}\end{matrix} ) + \\ {( 1 - {λ}_{j} )}^{i - 1} \cdot {ɛ}_{1 , j} , i \geq 2\end{matrix} \\ {ɛ}_{1 , j} = {ɛ}_{0 , j} = 0\end{matrix} \qquad Eq . 9 ''$$

The weights

$$λ \cdot {\sum }_{k = 2}^{i} {( 1 - {λ}_{j} )}^{i - k}$$

$$\{ \begin{matrix}{ɛ}_{i , j} = \begin{matrix}{λ}_{j} \cdot {\sum }_{k = 2}^{i} {( 1 - {λ}_{j} )}^{j - k} \cdot ( \begin{matrix}{V}_{k - 2 , j} - \\ {\hat{V}}_{k - 2 , j}\end{matrix} ) + \\ {( 1 - {λ}_{j} )}^{i - 1} \cdot {ɛ}_{1 , j} , i \geq 2\end{matrix} \\ {ɛ}_{1 , j} = {ɛ}_{0 , j} = 0\end{matrix} \qquad Eq . 9 ''$$

decrease geometrically with the age of the error. Furthermore, the weights sum to unity, since:

$$\begin{matrix}{λ}_{j} \cdot {\sum }_{k = 2}^{i} {( 1 - {λ}_{j} )}^{i - k} = {λ}_{j} \cdot {\sum }_{k = 0}^{i - 2} {( 1 - {λ}_{j} )}^{k} \\ = {λ}_{j} \cdot \frac{1 - {( 1 - {λ}_{j} )}^{i - 3}}{1 - ( 1 - {λ}_{j} )} \\ = 1 - {( 1 - {λ}_{j} )}^{i - 3}\end{matrix} \qquad Eq . 10$$

Let

$$λ = [ \begin{matrix}{λ}_{1} & 0 & ⋯ & ⋯ & 0 \\ 0 & ⋯ & ⋯ & ⋯ & ⋯ \\ ⋯ & ⋯ & {λ}_{j} & ⋯ & ⋯ \\ ⋯ & ⋯ & ⋯ & ⋯ & 0 \\ 0 & ⋯ & ⋯ & 0 & {λ}_{n}\end{matrix} ]$$

the diagonal matrix of the weights of each component and

$${ɛ}_{i} = [ \begin{matrix}{ɛ}_{i , 1} \\ ⋯ \\ {ɛ}_{i , j} \\ ⋯ \\ {ɛ}_{i , n}\end{matrix} ] .$$

Using the above notations, Eq. 8″ becomes:

$$\{ \begin{matrix}{ɛ}_{i} = λ \cdot ( {V}_{i - 2} - {\hat{V}}_{i - 2} ) + ( I - λ ) \cdot {ɛ}_{i - 1} , i \geq 2 \\ {ɛ}_{0} = {ɛ}_{1} = 0\end{matrix} \qquad Eq . 11 ”$$

εᵢ₋ₖ is recursively substituted:

$$\begin{matrix}\{ \begin{matrix}{ɛ}_{i} = {\sum }_{k = 2}^{i} {( I - λ )}^{i - k} \cdot λ \cdot ( {V}_{k - 2} - {\hat{V}}_{k - 2} ) + {( I - λ )}^{i - 1} \cdot {ɛ}_{1} , i \geq 2 \\ {ɛ}_{0} = {ɛ}_{1} = 0\end{matrix} & Eq . 12 ”\end{matrix}$$

Thus, the corrected volume at time tᵢ can be calculated as follows:

$${\hat{V}}_{i} = {A}^{- 1} \cdot [ ( ( 1 - {α}_{i} ) \cdot {\sum }_{k = 0}^{i - 1} {r}_{k} Δ t - {α}_{i} \cdot {C}_{0} ) {V}_{BT} + ( {\sum }_{k = 0}^{i - 1} {δ}_{k} Δ t + {r}_{0} + {\sum }_{k = 0}^{i - 1} {r}_{k} ) Δ t \cdot {V}_{PT} ] \pm \vert {ɛ}_{i} \vert \qquad Eq . 13 ”$$

In equation 13″, absolute value of error is added when under dosing occurred at previous time step, otherwise value of error is subtracted when over dosing occurred at previous time step.

As seen above, the algorithm determines a rate of depletion over a depletion time period of at least one of the additives in the chemical composition based upon a concentration of the additive measured by the monitor in comparison to a concentration of the additive previously measured by the monitor. The algorithm also predicts an amount of at least one additive that when added to the chemical composition will maintain a concentration of the additive within a predetermined concentration range around a predetermined setpoint concentration. The algorithm also corrects the predicted amount by adding to, or subtracting from, the predicted amount, an amount of the additive based upon previous concentrations of the additive measured by the monitor. The corrected amount is the dose added to the chemical composition by the dosage element.

In contrast to prior art controllers, the algorithm has the following additional novel features. The amount added to or subtracted from the predicted amount is based upon at least two previous measurements by the monitor of the concentration of the at least one additive. Also, the time interval between additions of the at least one additive is less than a time interval between measurements by the monitor.

Furthermore, the time delay intervals between additions is also adjustable, i.e., it may be varied to any interval no less than the time it takes the dosage element to add the at least one additive. Moreover, the predicted amount is also based upon a derivative of the depletion rate. Finally, the predicted amount does not depend upon a variable condition at the chemical containing unit.

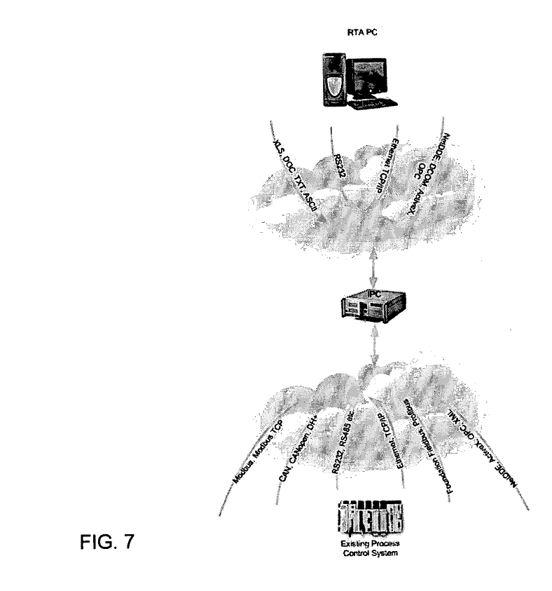

FIG. 7 is a schematic illustrating existing process control systems and existing computerized system capabilities. As shown in FIG. 7, the solution breaks down into hardware and network devices, industrial communication protocols and software applications.

A virtual sensor for monitoring the composition of electroplating bath solutions containing organic additives which can be retrofitted to existing equipment is also described. Specifically, in the case of the RTA integration an apparatus and method were developed to solve the lack of communications and/or communication problems between the real time analyzer (RTA) which is typically housed in a personal computer and the process control system. Current “real time” analyzers typically take from 10 to 25 minutes or more to perform an analysis and to obtain concentration data. As technological advancements are made, it will likely take less time to obtain measurement data, making such measurements closer to actual real time conditions. Thus, this invention can be used to better control bath conditions between “real time” measurements.

The present invention also describes a virtual sensor concept that provides automatic, real-time additive concentration using classical prediction techniques detailed hereafter. The sensing technology does not require costly analyzers nor expensive hardware to operate, and it also allows to close the control loops in a much more reliable and deterministic way. Real-time feedback control becomes thus possible and additive concentrations in plating baths can then be maintained using the most adequate process control strategies.

The sensor is based on an artificial intelligence system that automatically computes additive concentrations in efforts to compensate for the lag time in getting concentration readings from real-time monitors, using measurable process parameters. Such sensing system allows the easy implementation of a continuous, uninterrupted process control strategy to maintain the physical and/or chemical state and quality of liquid baths such as those using into plating operations at all times. The virtual sensor proposed in this invention will estimate the state of the electroplating process, which is a discrete time controlled process.

The sensor will provide real-time estimates of the additive concentrations while minimizing their prediction errors. The real-time additive concentrations can readily be used to control plating solution composition. It is therefore possible to continuously calculate the amount of additives to inject into the process and maintain bath consistency. The virtual sensor of this invention calculates and minimizes prediction errors while allowing real-time bath composition control. It uses well-proven, powerful mathematical and process control techniques. Prior to this invention, such techniques have not been used in association with electroplating baths.

In a non-limiting embodiment, the state of the electroplating process is determined by the concentration of chemicals in the electroplating bath, and the concentration of such chemicals are monitored and controlled by this invention. Of course, the state of the electroplating process can also be determined by any other parameters which can be used to determine the state of the electroplating process.

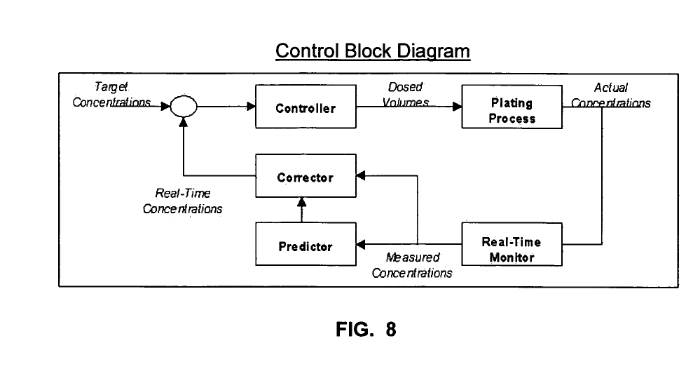

This concept can be illustrated by the control block diagram of FIG. 8. FIG. 8 shows that real time concentrations are compared with the target concentrations with the difference or deviation, if any, being sent to the controller. The controller then determines and/or implements the dosed volumes of a certain chemical concentration added to the bath such as during the plating process. The actual concentrations within the plating bath are measured in a real-time analyzer to determine the actual measured concentrations of the chemicals/additives. This information is sent to a computer which serves as a predictor and corrector and provides the real time concentrations that are again compared with the target concentrations in the feedback loop. Upon comparison of the real time concentrations with the target concentrations further adjustments in the chemistry of the bath can be made, if necessary. If the difference or deviation is small and the concentrations are still within the desired predetermined range, and is considered to be insignificant, dosing may not occur. This will depend upon the parameters that are used within the controller for dosing. Also in an embodiment, the real time concentrations can be used to alter the predicted concentrations. This would especially be useful for baths during start-up operations and/or shutdown operations, when manufacturing operations are on stand-by for certain time periods, or when manufacturing conditions are changed (e.g. increased or decreased production).

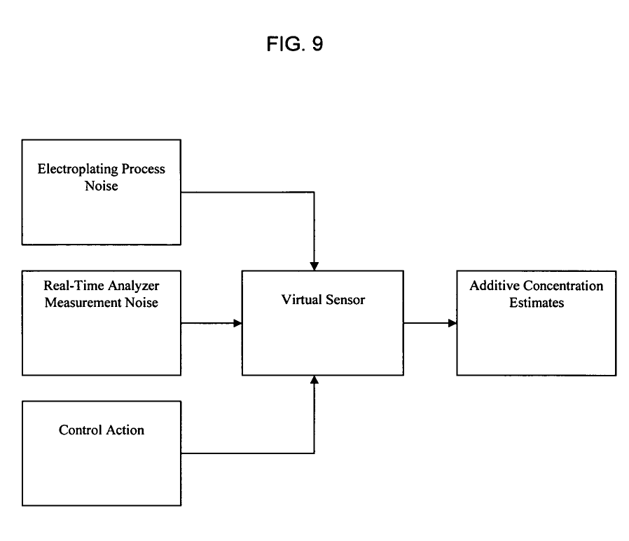

A non-limiting embodiment of a filter based virtual sensor for electroplating bath monitoring and control is shown in the schematic of FIG. 9. As shown in the illustrative schematic of FIG. 9, one or more inputs of information such as electroplating process noise, real-time analyzer measurement noise, and/or control action are transmitted to the virtual sensor, and based upon such inputted information, additive concentration estimates are determined, and if appropriate result in the dosing of one of more chemicals into the bath. More specifically, the virtual sensor will estimate the additive concentrations (both the organics and inorganics) at a given time then obtain real (noisy) measurement from a real-time analyzer. The equations governing the virtual sensor are divided in two categories: time update and measurement update equations. The time update equations are used to project forward in time the current state, typically additives concentrations, and error covariance estimates to obtain the a priori estimates for the next time step. Conversely, the measurement update equation will incorporate a new measurement into the a priori estimate to obtain an improved a posteriori estimate. The time update equations can be thought of as predictor equations, while the measurement equations can be thought of as corrector equations. The data output of the filter can be passed to the process controller for dose calculation and implementation. The calculated dose can be a potential dose or a dose which is actually implemented.

Since the sensor is in fact a mathematical algorithm, it can be written using any program development language (such as C/C++ alike) and compiled into executable format and is used in the form of software installed on the control platform of interest, be it a personal computer, industrial computer, a programmable controller, a distributed control system or any hardware for process control.

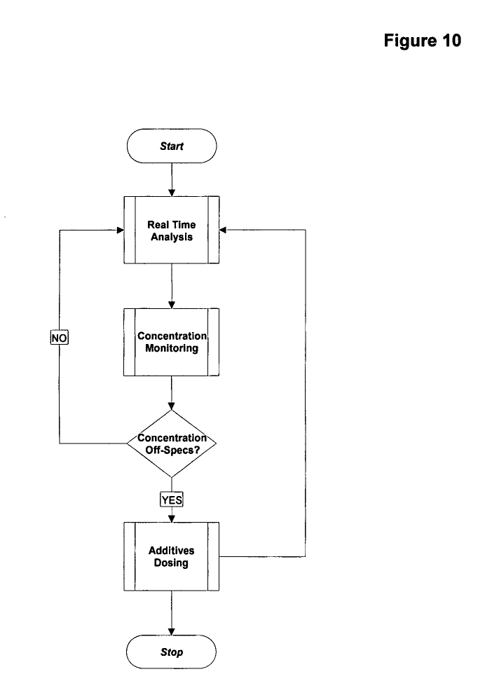

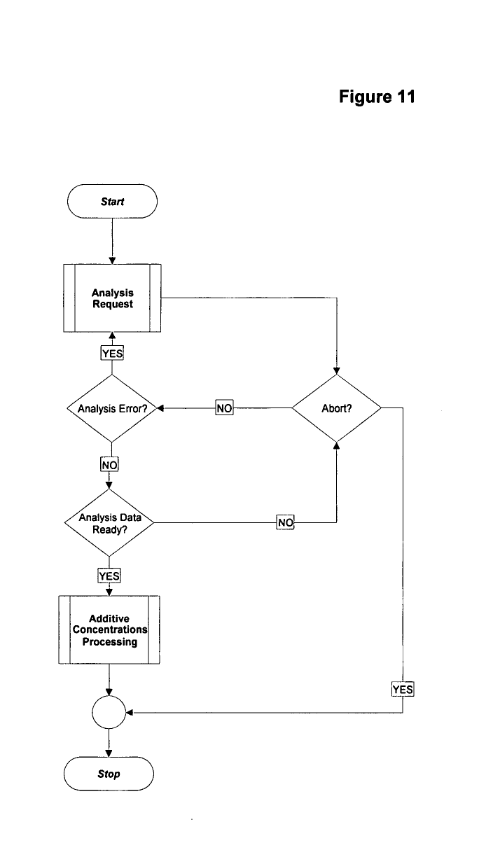

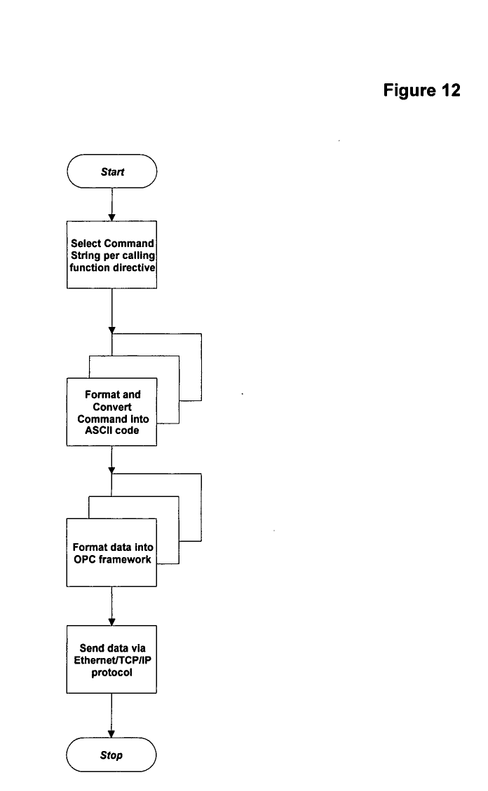

A variety of algorithms can be used to implement the control scheme. FIGS. 10-15 are non-limiting embodiments of illustrative flow charts showing the program or code functions that can be used. The various program functions are shown in several boxes and other differently shaped forms and the boxes and other shaped forms have different meanings. For example, the rectangles that have edges with double vertical lines, such as those in FIG. 10 designate steps which are major functions and rely upon macrofunctions which will be detailed later in a one or more functions. The diamond shaped boxes refer to functions/steps where a decision and/or inquiry are initiated, which in FIG. 10 contain an inquiry as to whether the concentration is within or off-specification. A further circular shape such as shown in FIG. 11 entitled Real Time Analysis System Flow Chart of Program Functions is a connector or connection point, with no specific meaning. Further shapes are shown in FIG. 12, such as a plain rectangle which designates a low-level function, which reports data back to a higher level function, such as that shown in the rectangles with double vertical lines edges, and lack other levels of functions in the program structure. The partially superimposed or nested boxes and rectangles refer to functions that occur in parallel, rather than in series, such as that shown in FIG. 12 which is entitled Analysis Request Flow Chart of Program Functions.

FIG. 10 is a non-limiting schematic illustrating an embodiment of the invention showing the main flow chart of program functions designated for convenience as “Real Time Analysis,” “Concentration Monitoring,” and “Additives Dosing” which designate steps in the control scheme and are major functions that rely upon macrofunctions which will later occur. The function denoted as real time analysis, leads the analyzer through a set of instructions or commands. It processes the signal it receives from the instrument which indicates a value for the characteristic being measured, such as concentrations, and converts the signal it into useful data that is then passed on to the “concentration monitoring” function. The function denoted as “concentration monitoring” calculates additives concentrations in the bath management system and the process tool using real time analysis data and a predictive corrective algorithm. This function can be adjusted to continuously calculate the additives concentrations, or if desired, at other intervals of time which are determine to provide sufficiently accurate data. The information from the concentration monitoring function is sent to the next function which requires a decision and/or initiates an inquiry to determine whether the concentration is within specifications. Here, each predicted additives concentration is compared to its operating set point, also known as specification, which in an embodiment can also be a concentration range. If any concentration becomes outside its target by a predetermined amount, a dosing cycle will be initiated to correct the concentration of such additive. The dosing cycle is performed by a programmable logic controller, and the system directly or indirectly causes the physical addition of a dose of at least one chemical, and/or a diluent, into the electrochemical bath or electrochemical solution, blends the solution, and transfers the solution to the electro copper plating tool, or another desired location. Thereafter, the program resumes the real time analysis function. Conversely, if the concentration is not off-specification (within specification), the next step is a return to the real time analysis function. The time delay between each program function can be chosen and changed, as necessary. Further, it should be contemplated there can be more than one set of predicted operating conditions for differing rates of manufacturing. For example, if production must be ramped up to fill special orders, the rate of depletion would be increased and to compensate adjustments must be made. As further example, if there is one rate of production during normal business hours and another rate of production after-hours, the rate of depletion and dosing must again be adjusted. This invention contemplates that “normal operating conditions” can vary and that there can be a database of stored predicted operating conditions from which to choose. Additionally, the predictor data that are stored in memory can be further adjusted based upon actual conditions that are fed back to the controller.

FIG. 11 is related to FIG. 10 and is a schematic further illustrating the program or code functions of the real time analysis algorithm. Herein, a function denoted as the analysis request function will send commands to from the remote Industrial Computer to the RTA computer system. Once this occurs, if this function is then is aborted by a user or a calling function, the analysis will be stopped. If the function is not aborted, an analysis is performed. In an embodiment, one or more analyses of one or more chemicals can be initiated by this function. Once such analytical data is obtained, an inquiry is initiated to determine whether the analysis results are erroneous (i.e., corrupted data, incomplete cycle, etc.). If the results are determined to be faulty, the program will re-initiate a new command analysis. Conversely, if there is no error, then an inquiry is made as to whether the analysis data is ready. The calling function will be scrutinizing for incoming data stream (analysis results) from the RTA system. This step is a passive one, i.e. triggered on communication request from the client application. If the data is not ready when this occurs, the function is terminated. However, if the data is ready, it is forwarded to the next function, designated for convenience as additive concentrations processing. There, the incoming data stream is filtered and processed by this program and made available to the calling function, which is typically, the “concentration monitoring” function. However, the data stream could be made available to another calling function. Once the additives concentration processing has occurred, or the analysis aborted, the program functions will be re-started with the analysis request. Again, the time delay between each program functions can be chosen and changed, as necessary.

FIG. 12 is related to FIG. 10 and is a schematic further illustrating the program functions for requesting analysis. The purpose of the first command of this program is to select command string per calling function directive. This function will send a RUN command to the analyzer system. The syntax is: RUN Bath_Code, Number_of_Runs, Time_Between_Consecutive_Runs. The calling function may also request that one or more of the following commands be sent:—STOP, PAUSE, RESUME, SKIP Run_Number_x, REDO Run_Number_x, REDO ALL, and CANCEL CURRENT RUN. Similar calling functions and syntax could be used which cause the same actions. Once one or more of these commands are sent and executed, there are nested functions for formatting and converting commands into ASCII code. The nested boxes indicate that the function is executed in a parallel function, rather than in series. This multi-sequence function will frame the string commands and convert them into ASCII code. Next, the data is directed into an OPC framework. There, the function will frame the data in such a way that it can be exchanged using the OLE for Process Control open connectivity standard. Following this, data is sent via Ethernet/TCP/IP protocol. After this function occurs, the string of commands is resumed at the beginning. As before, the time delay between each program functions can be chosen and changed, as necessary.

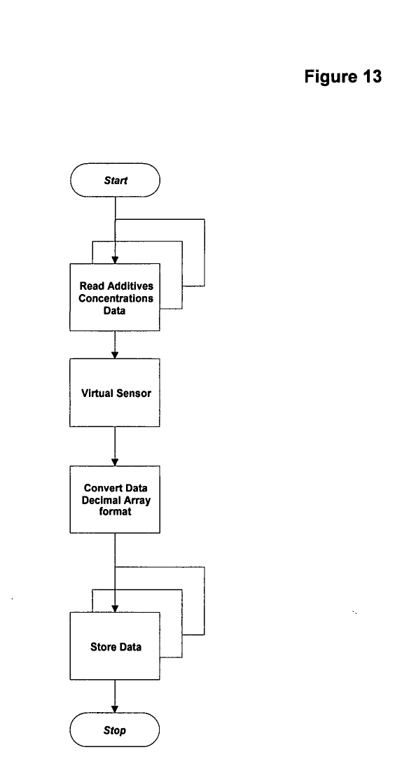

FIG. 13 is related to FIG. 10 and is a schematic further illustrating the program functions for additives concentration processing. Once the program is started, the read additives concentrations data function extracts incoming data from the TCP/IP framework and is converted into useful data for the program. The program filters the data from any noise, using a band filter. The concentration data is then stored in temporary memory registers in the computerized control system such as an industrial PC. The virtual sensor concentration data is then filtered using the virtual sensor algorithm of this invention. The virtual sensor is based on the Kalmann Filter principle. Corrected data is then stored in temporary or permanent memory registers (archived) in the computerized control system.

Next, the convert data decimal array format function converts data into double floating points and concatenated, or connected, into a matrix, whose dimensions are the discrete time index and number of additives. The store data function stores the data matrix into dedicated or addressed registers for future use. A calling function such as “concentration monitoring” has access to those registers. After this function occurs, the string of commands begins again. Again, the time sequence between each program functions can be chosen and changed, as necessary.

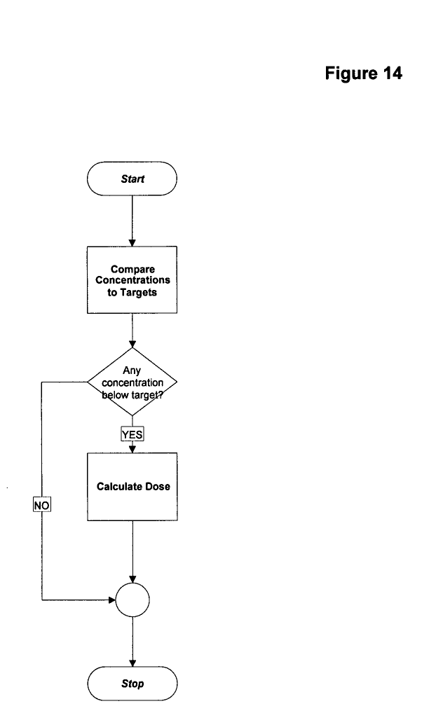

FIG. 14 is related to FIG. 10 and is a schematic illustrating the program functions for concentration monitoring. First, the function compares the concentrations to targets. This function retrieves the data matrix at time t (current) and computes the differential between the target and current filtered and corrected concentrations. The resulting Matrix will be used to assess the deviation from target concentrations. Next an inquiry is made to determine whether there is any concentration outside of the target. For each additive, the differential is compared to a predetermined scalar, referred to as deviation. The deviation is formulated as percentage of the target concentration of that particular additive. This function will determine whether the differential at time t is negative and greater than the deviation in absolute value. If it is determined that the measured concentration is outside of the target, which typically would be below the target, this function computes the amount of the chemical additive to add for each additive which was deemed to be depleted to bring its concentration within the normal operating range in the electroplating bath. The calculation is performed using the predictive corrective algorithm. Each dose is then stored in a memory register. The function also informs the calling function that concentrations are “off-spec” or outside specification by switching on an off-spec indicator. The off-spec indicator is then capable of effecting a visual or audio alarm, or transmitting a signal to alert an operator such as through a pager or an intranet, the Internet. However, if the measured chemical is within acceptable concentration limits that are within the predetermined concentration range, no action is taken. Thereafter, the program functions are re-started.

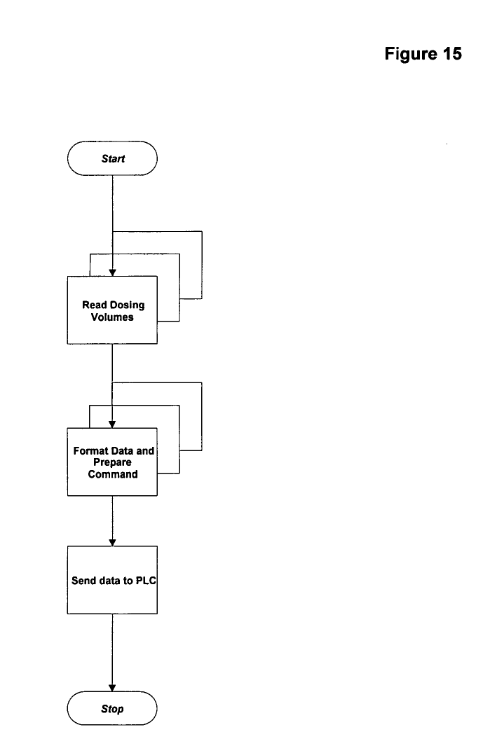

FIG. 15 is related to FIG. 10 and is a schematic illustrating the program functions for additives dosing. The read dosing volumes function reads the dosing volumes values from their memory registers in the IPC when the off-spec indicator is “ON”. The nested boxes indicate this function operates in parallel. The dosing volumes data is conveyed to the next function which formats data and prepares commands. More specifically, this function will convert each dosing volume into double floating points. For each additive that should be added to bath (i.e. a corresponding non-zero volume) this program will select the appropriate command to instruct the PLC to proceed with the dosing cycle. The command is digitized so that a PLC program can make use of it. The string of commands is then coded in a Hexadecimal format. This information is then conveyed so that the next function operates to send data to the PLC. This function will frame the data in such a way that it can be transported over an Ethernet backbone using a protocol compatible with the PLC (e.g. Modbus TCP).

Thus, one non-limiting embodiment of the method includes the following steps. An analyzer measures a property of a chemical bath. The chemical bath's location includes, but is not limited to, one or several semiconductor tools, a blending tank, conduits therebetween, a combination of two or more of the foregoing locations, as well as anywhere in the circuit where measurements are desired. The analyzer transmits a first data signal corresponding to the measured properties to a first computerized control system using a proprietary communication protocol, specific to the analyzer, and which may vary from one analyzer to another, and is therefore considered to be non-standard. The first computer transmits a second data signal corresponding to the first data signal to a computerized process control system using the proprietary communication protocol, wherein in a non-limiting embodiment the later is an industrial PC. A combination of hardware and software allows the industrial PC to translate the second data signal using the proprietary communication protocol to a third data signal using an open communication protocol, which could be considered to be a universal-type protocol which is not proprietary. In a non-limiting embodiment, the standard communication protocol is a protocol that can be considered to be universal to a particular communication protocol. In a non-limiting embodiment, the standard communication protocols are selected from those that can be used with software that is commonly used in computer operating systems, such as Windows-based software (Microsoft Corporation). Obviously, other operating systems, known to one skilled in the art, may be used on the computers. The third data signal is transmitted to a control system. The control system controls modification of a property, which in a non-limiting embodiment is concentration of one or more chemicals in the electroplating bath, as well as other measured locations. In a non-limiting embodiment, the measured properties include concentration of chemical, temperature of solution, pH of solution, as well as any other measurable chemical or physical property. In a non-limiting embodiment, the control system controls one or more of the following tasks: addition of one or more chemical component(s) to the chemical bath, draining of a portion of the chemical bath, and commencement of the measurement of at least one property of at least one location of the chemical bath, chemical component, input to the tool and/or output from the tool by the analyzer. In a further non-limiting embodiment, as should be contemplated, one or more measurements can be done in one or more locations of the chemical bath(s) by employing a plurality of analyzers.

In a non-limiting embodiment, the control system controls the analyzer by transmitting a fourth data signal using the standard communication protocol to the second computer, wherein a combination of hardware and software allows the second computer to translate the fourth data signal to a fifth data signal using a proprietary communication protocol which is transmitted to the first computer, and wherein the first computer transmits a sixth data signal to the analyzer using the proprietary communication protocol requesting commencement of measurement of the property of the chemical bath.

The retrofit includes networking the real-time analyzer (RTA) PC and the control system using a communication protocol such as an Industrial Ethernet protocol or a NetDDE or OPC, or the like. OPC stands for OLE for Process Control, and is a standard developed in 1996 by an industrial automation industry task force, and specified the communication of real-time plant data between control devices from different manufacturers. The original standard was designed to bridge Windows based applications and process control hardware and software applications. It is an open standard that permits a consistent method of accessing field data from plant floor devices. As should be contemplated, new systems can also be made which embody the invention disclosed herein which would not require retrofitting, and this disclosure is intended to cover such systems. As such, in a non-limiting embodiment, the controller and analyzer could be packaged together, with each component being separate, or if desired in one console.

OPC servers provide a method for many different software packages to access data from a process control device, such as a PLC or DCS. Traditionally, any time a package needed access data from a device, a custom interface, or driver, had to be written. The purpose of OPC is to define a common interface that is written once and then reused by any business, SCADA, HMI, or custom software packages. Once an OPC server is written for a particular device, it can be reused by any application that is able to act as an OPC client. OPC servers use Microsoft's OLE technology (also known as Component Object Model, or COM) to communicate with clients. COM technology permits a standard for real-time information exchange between software applications and process hardware to be defined. There is also a newer version of OPC protocol that also uses XML for communications.

In a further non-limiting embodiment, a secure connection can be used to communicate the signals, using any physical Ethernet backbone or wireless radio frequency (RF) media. In another non-limiting embodiment, a USB serial interface or wireless technology such as a Bluetooth™ can be used to interface with the PC. In a further non-limiting embodiment, wireless technology can be used to transmit the information to an intranet, the Internet, or the Ethernet.

An Industrial Computer will be the connection between the RTA PC on the one hand, and the process control system on the other. As used herein, the term Ethernet is meant to refer to a frame-based computer networking technology for local area networks (LANs).

In a non-limiting embodiment, a modified network card is used in the first computer to avoid conflicts between the data signal from the analyzer and automatic actions that are performed by a network card conventionally used with the first computer, wherein the conflicts result in the first computer's failure to recognize communication at any of its ports, including that which communicates with one or more other personal computers, such as a second computer.

Hardware and Network Devices:

In a non-limiting embodiment, the RTA, like most analyzers used for bath monitoring is controlled and programmed via a personal computer (PC) in its stand-alone configuration. Most PCs support serial communication via their built-in serial port (RS-232). Unfortunately, the traditional RS-232 communication is slow (9600 being a typical baud rate) compared to the state-of-the-art industrial networks, and limited in distance (10 to 15 meters). RS-232 is a standard for serial binary data interconnection between a DTE (Data terminal equipment) and DCE (Data communication equipment). It is commonly used in computer serial ports. A similar ITU-T standard is V.24, which is a list of definitions for interchange circuits between data terminal equipment (DTE) and data circuit terminating equipment (DCE). This is not to mention that only peer-to-peer topologies are possible. RS-232 is gradually being replaced in personal computers by USB for local communications. USB is faster and has connectors that are simpler to connect and use, and has software support in popular operating systems. Further, USB is designed to make it easy for device drivers to communicate with hardware, and there is no direct analog to the terminal programs used to let users communicate directly with serial ports.

Most of today's PCs are equipped with a Network Interface Card and do support the IEEE 802.3 Ethernet standard communication protocol. Baud rates of 100 mega bytes per second and even giga bytes per second are achievable. This networking solution also offers flexible topologies such as bus configurations ideal for Local Area Networks.

On the other hand, the existing process control system may be present in proprietary hardware with limited communication capabilities. Control systems are traditionally running over Distributed Control Systems (DCS), Programmable Logic Controllers (PLC) or Industrial PCs (IPC). It is now frequent to find these systems Ethernet enabled, via the addition of a hardware specific Ethernet communication card.

In order to interface an RTA and the Controller, an IPC layer can be used therebetween. The IPC can support both serial, proprietary (when loaded with adequate drivers) and Ethernet communications and can therefore act as a bridge between the RTA PC and the prospective proprietary or non-standard control system hardware and software. Additionally this invention can be used to interface a proprietary control system hardware and software to a RTA with the control system hardware and software that may or may not be proprietary. In a non-limiting embodiment, the devices will be connected to one another in a bus configuration. An industrial Ethernet Switch complements the network topology. Router or Gateways may be added to the initial configuration if the LAN is to be integrated to a WAN or a Corporate Network. Of course this system can also be used in conjunction with a variety of IP nets, which in a non-limiting embodiment includes the Internet, intranet and extranet, and other hardware and/or software can be used to further interface and/or integrate the components.

Communication Protocol:

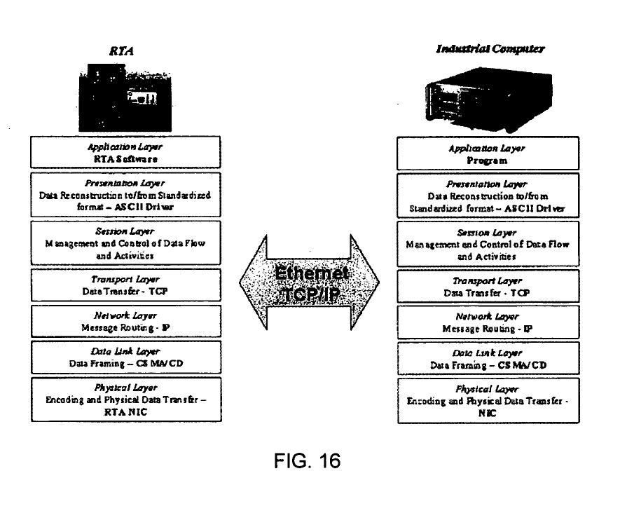

FIG. 16 is a schematic showing the interface of an RTA and industrial PC, utilizing the ISO OSI seven-layer communication model. The selected protocols are in compliance with the ISO OSI seven-layer communication model. As shown in FIG. 16, the protocols utilized at each layer are as follows:

The Data Link Layer uses the Carrier Sense Multiple Access/Collision protocol. This layer establishes physical connection between the local machine and the destination. This is done in hardware with application specific integrated circuits (located on the NIC).

The Network Layer uses the Internet Protocol. The IP protocol routes data from node to node by opening and maintaining the appropriate path. This is handled in software.

The Transport Layer uses the Transmission Control Protocol, which performs error checking and controls transmission by ensuring data integrity through the message structure protocol. This is handled in software.

The Session Layer creates and maintains communication channels and handles security and login. In a non-limiting embodiment, the layer is absent, as a dedicated LAN is used.

The Presentation Layer converts received data into a designated Human Machine Interface. In a non-limiting embodiment, data is converted into ASCII format for transmission to the remote side. This is handled in software.

The Application Layer is the program running on the RTA PC, also referred to as user interface. The IPC may or may not need an application layer depending on whether local access is desirable or not.

Software:

The RTA user interface program needs to be coupled with a specific application to serve up data to a client application. This server application may be directly installed on the RTA PC and the two programs can exchange data via DDE, ActiveX etc. Otherwise, this application will be installed on the IPC and the two programs will exchange data via NetDDE or DCOM. As used herein, the term DCOM is an abbreviation for Distributed Component Object Model, and is a Microsoft proprietary technology for software components distributed across several networked computers to communicate with each other. As used herein, the term NetDDE is a Windows facility which enables DDE messages to be sent between applications running on different machines. As used herein, Dynamic Data Exchange (DDE) is a technology for communication between multiple applications under Microsoft Windows and also OS/2. Although still supported in even latest Windows versions, it has mostly been replaced by its much more powerful successors OLE, COM, and OLE Automation. DDE allows one application to open a session with another, send commands to the server application and receive responses. A common use of DDE is for custom developed applications to control off-the-shelf software.

Similarly, the server application on the IPC will have to exchange data with the existing process control system. A flexible solution is to use and OPC server. Such solutions are typically readily available in the form of out-of-the-shelf packages, based on the specific process control hardware already in place. The OPC server will be installed and configured to run on the IPC. This integration scheme seems to be the most adequate considering the new connectivity standards and best practices in process control. It is nonetheless possible to achieve connectivity between the IPC and the existing process control system using other Fieldbus technologies or conventional communication protocols (Modbus, Modbus PLUS, CAN, Profibus, Foundation Fieldbus etc.).

In a further aspect of this invention is based on a Kalman Filter concept algorithm, which estimates the process state at some point in time and then obtains feedback in the form of noisy measurements preferably through a real-time analyzer.

Process Estimate Formulation:

In this non-limiting embodiment, it is assumed that the additive concentration degradation due to the electroplating is governed by linear stochastic equations of the following type:
Cₖ=ACₖ₋₁+Buₖ₋₁+Wₖ₋₁ Eq. 14
Mₖ=HCₖ+vₖ Eq. 15

In the equations above, Cₖ, Mₖ, and vₖ are the actual additive concentrations, the measured additive concentrations, and the measurement noise at time step k. uₖ₋₁, wₖ₋₁ the control action (optional) and process noise at the previous time step k-1. Process and measurement are assumed to be independent, white, and with normal probability distributions, which is the case in most chemical processes. In other words:
p(w)˜N(0, Q) Eq. 16
p(v)˜N(0, R) Eq. 17

Q and R being the process noise covariance and measurement noise covariance respectively.

The n×n matrix A relates the concentrations at the previous time step k-1 to the concentrations at the current time step k, in absence of either a driving force (control action, i.e. additive dosing) or process noise. The n×l matrix B relates the optional control actions to the concentrations. The m×n matrix H relates the actual concentrations to the measured concentrations.

Finally, the size of the previous matrices can be interpreted as follows: n is the number of additives and/or process state variables, m the number of measurements taken at a given time step, and l the number of manipulated variables used to control the process.

Prediction Errors Formulation:

Ĉₖ⁻ is the a priori concentration estimate at time step k given knowledge of the process prior to step k, and Ĉₖ is the a posteriori concentration estimate at time step k given measurement Mₖ. The a priori and a posteriori estimate errors can be defined as eₖ⁻=Cₖ−Ĉₖ⁻; [Eq. 18] and eₖ=Cₖ−Ĉₖ [Eq. 19], respectively. Therefore, the a priori estimate error covariance and a posteriori estimate error covariance are defined as Pₖ⁻=E└eₖ⁻eₖ^{-T}┘ [Eq. 20] and Pₖ=E└eₖeₖ^{T}┘ [Eq. 21], respectively.

Blending Factor Formulation:

The objective of the virtual sensor is to compute the a posteriori concentration estimate Ĉₖ as a linear combination of an a priori estimate Ĉₖ⁻ and a weighted difference between an actual real-time analyzer measurement Mₖ and measurement prediction HĈₖ⁻:
Ĉₖ=Ĉₖ⁻+Kₖ·(Mₖ−HĈₖ⁻) Eq. 22

The n×m matrix K is called the gain or blending factor. It is chosen so that it minimizes the a posteriori error covariance Pₖ. It can be shown using the definition of the a posteriori error, and the above equation, and solving for K that the blending factor is given by the following expression:
Kₖ=Pₖ⁻H^{T}(HPₖ₋₁⁻H^{T}+R)⁻¹ Eq. 23
where Pₖ⁻=A^{T}Pₖ₋₁A^{T}+Q Eq. 24
Virtual Sensor Parameters and Tuning:

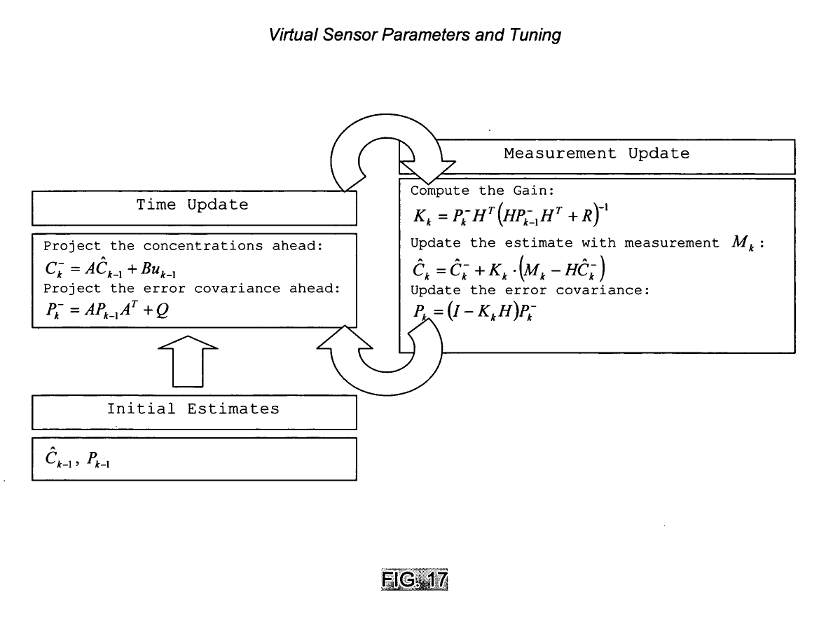

The principle of the virtual sensor described in this invention has some of the same features of the discrete Kalman Filter, and is depicted in FIG. 17, with the time update and measurement equations being previously presented in the foregoing paragraphs.

The measurement noise covariance R is usually measured prior to implementation of the virtual sensor. It can be done using offline sample concentration measurements as to determine the variance of the measurement noise.

The computation of the process noise covariance is more complicated as current technology does not allow for the direct observation of the process which is the subject of attempted to estimation. In a non-limiting embodiment of a virtual sensor, a “poor” process model is recommended such as a linearization around a known steady-state operating condition, for example 1^{st} or 2^{nd} order Taylor Expansion, to inject enough uncertainty into the process through the selection of Q.

In the foregoing specification, the invention has been described with reference to specific embodiments thereof, and has been demonstrated as effective in providing methods and apparatus for retrofitting existing control systems and thereby providing virtual sensors with improved real time monitoring and control capabilities. However, it will be evident that various modifications and changes can be made thereto without departing from the scope of the invention as set forth in the appended claims. Accordingly, the specification is to be regarded in an illustrative rather than a restrictive sense. For example, specific combinations of control and/or analyzing apparatuses that fall within the claimed parameters, but which are not specifically identified or illustrated herein, are anticipated and expected to be within the scope of this invention. Also, the cited examples of chemicals that are added, used and/or depleted during electroplating operations or which are capable of being monitored and/or controlled are merely illustrative and not intended to limit the scope of useful chemicals for the various stages of the present process.

Having described novel systems and methods for monitoring, dosing and distribution of chemical compositions in material treatment processes, it is believed that other modifications, variations and changes will be suggested to those skilled in the art in view of the teachings set forth herein. It is therefore to be understood that all such variations, modifications and changes are believed to fall within the scope of the claims.

What is claimed is:

1. A method for controlling concentration of a chemical in a liquid bath during manufacturing, said method comprising:
a) predetermining a range of acceptable concentrations for one or more chemicals used in the bath during manufacturing;
b) using an analyzer to measure concentration of said one or more chemicals in the bath, wherein said measured concentration comprises a first data signal having a proprietary communication protocol;
c) transmitting the first data signal that corresponds to the measured concentration from the analyzer to a first computer;
d) causing the first computer to transmit a second data signal corresponding to the first data signal to a second computer, said second signal having a proprietary communication protocol;
e) using hardware and/or software to cause the second computer to translate the second data signal having a proprietary communication protocol to a third data signal having an open communication protocol; and
f) transmitting the third data signal to a controller that is capable of controlling concentrations of one or more chemicals in the bath.

2. The method of claim 1, wherein said controller causes an addition of one or more chemicals to the bath.

3. The method of claim 1, wherein the controller causes draining of a portion of the chemical bath.

4. The method of claim 1, wherein said controller causes measurement of at least one chemical concentration in said bath.

5. The method of claim 4, wherein concentration is measured in one or more locations of the chemical bath(s) by using one or more analyzers.

6. The method of claim 1, further comprising the step of using the controller to control the analyzer by transmitting a fourth data signal having an open communication protocol to the second PC, wherein hardware and/or software enables the second PC to translate the fourth data signal to a fifth data signal having a proprietary communication protocol.

7. The method of claim 6, further comprising the step of transmitting the fifth data signal to the first computer, wherein the first computer transmits a sixth data signal to the analyzer having a proprietary communication protocol.

8. The method of claim 7, wherein the sixth data signal causes measurement of the concentrations of one or more chemicals in said bath.

9. The method of claim 2, wherein said controller comprises a processor programmed with an algorithm, wherein said algorithm is written into a program development language and compiled into an executable format.

10. The method of claim 9, wherein said algorithm enables computation of an initial concentration estimation, a time update for the initial concentration estimation, and a measurement update.

11. The method of claim 10, wherein said initial concentration estimation is determined by:
Ĉₖ₋₁, Pₖ₋₁
where Ĉₖ⁻ is an a prior concentration estimate at time step k considering a process step preceding step k, and Pₖ is an a posteriori concentration estimate at time step k considering a process step preceding step k.

12. The method of claim 10, wherein the time update for the initial concentration estimation is determined by:
a) projecting an a priori concentration, using
Ĉₖ⁻=AĈₖ₋₁+Buₖ₋₁ and
b) projecting an a priori error covariance, using
Pₖ⁻=APₖ₋₁A^{T}+Q.

13. The method of claim 10, wherein the measurement update is compared with the initial concentration estimate by:
a) computing the gain, using:
Kₖ=Pₖ⁻H^{T}(HPₖ₋₁⁻H^{T}+R)⁻¹;
b) updating the initial estimate by using actual concentration measurements Mₖ, using:
Ĉₖ=Ĉₖ⁻+Kₖ·(Mₖ−HĈₖ⁻);
c) updating error covariance, using:
Pₖ=(I−KₖH)Pₖ⁻; and
d) where Cₖ is actual chemical concentration, Mₖ is measured chemical concentration, and vₖ is measurement noise at time step k.

14. The method of claim 9, wherein said algorithm comprises a function for real time analysis, a function for concentration monitoring, a function for determining whether the concentration is within a predetermined specification, and a function for chemical dosing.

15. The method of claim 14, wherein said algorithm for real time analysis comprises a function for:
a) directing the analyzer through instructions or commands thereby causing said analyzer to output at least signal;
b) processing at least one signal from said analyzer, said signal conveying measurement data; and
c) converting said signal into data used for concentration monitoring.

16. The method of claim 14, wherein said algorithm for concentration monitoring, causes calculation of one or more chemical concentrations using real time analysis data and a predictive corrective algorithm.

17. The method of claim 16, wherein the algorithm initiates an inquiry to determine whether said one or more calculated concentrations are within acceptable predetermined concentrations.

18. The method of claim 14, wherein said algorithm for determining whether the concentration is within specification causes a comparison between a predicted chemical concentration and a predetermined operating concentration range or set point specification.

19. The method of claim 18, whereinin a dosing cycle is initiated if the concentration of such chemical is not within said range or specification.

20. The method of claim 14, wherein said dosing cycle is performed by a programmable logic controller, where the controller:
a) causes an addition of at least one dosage of chemical into an electrochemical bath solution;
b) causes blending of the bath solution; and
c) causes a transfer of said bath solution to a desired location.

21. A system for controlling concentration of a chemical in a liquid bath during manufacturing, said system comprising:
a) a predetermined range of acceptable concentrations for at least one chemical used in the bath during manufacturing;
b) an analyzer for measuring a concentration of at least one chemical in the bath, where said analyzer provides a first data signal comprising measured concentration data, said data signal having a proprietary communication protocol;
c) a first computer interfaced with said analyzer, where said first data signal is transmitted from said analyzer to said first computer and where said first computer transmits a second data signal corresponding to the first data signal to a second computer, said first signal having a proprietary communication protocol;
d) hardware and/or software capable of causing the second computer to translate the second data signal having a proprietary communication protocol to a third data signal having an open communication protocol; and
e) a controller capable of controlling the concentrations of said at least one chemical in said bath, where said third data signal is transmitted from said second computer to said controller and where said third data signal is used by said controller to control said concentration.

22. The system of claim 21, further comprising an algorithm used in said controller, where said algorithm is written into a program development language and compiled into an executable format, said algorithm acting as a virtual sensor to estimate target chemical concentrations at a given time, and said algorithm comprising equations for a time update and a measurement update.

23. The system of claim 22, wherein said controller is capable of commencing measurement of at least one chemical in said bath when said third data signal is transmitted to the controller.

24. The system of claim 23, further comprising a supply of said at least one chemical, where said controller controls said concentration in the bath by causing dosing of one or more chemicals to the bath and/or by draining a portion of the chemical bath.

25. The system of claim 23, wherein an estimated target concentration is compared with measured concentration data and wherein a difference between said target and said measured concentrations causes calculation of a potential and/or actual chemical dose for maintaining a predetermined concentration in said bath.

26. The system of claim 21, wherein one or more measurements are done in one or more locations of the chemical bath(s) by using one or more analyzers.

27. The system of claim 25, wherein the controller controls the analyzer by transmitting a fourth data signal having an open communication protocol to the second computer and where a combination of hardware and software enables the second computer to translate the fourth data signal to a fifth data signal having a proprietary communication protocol.

28. The system of claim 27, wherein the fifth data signal is transmitted to the first computer and wherein the first computer transmits a sixth data signal to the analyzer, said sixth signal having a proprietary communication protocol.

29. The system of claim 28, wherein the sixth data signal initiates measurement of the concentrations of one or more chemicals in said bath.

30. The system of claim 21, wherein the first and/or second computer is a personal computer or an industrial computer.

31. The system of claim 21, wherein said system is comprised of retrofitted analyzers and controllers.

32. The system of claim 31, wherein the retrofit is comprised of at least one networked real-time analyzer (RTA) computer and a controller and a communication protocol selected from the group consisting essentially of an industrial ethernet, a TCP/IP protocol, a NetDDE, an OPC server, or a combination of the foregoing.

33. A system for retrofitting a real-time analyzer (RTA) computer and a controller to improve real time control of chemical solutions used for material treatment process, said system comprising:
a) at least one networked real-time analyzer (RTA) computer, wherein said analyzer provides a first signal for transmitting data, said signal having a proprietary communication protocol;
b) a second computer for receiving said signal from said RTA, wherein said second computer converts said signal to a third signal having an open communication protocol;
c) a controller capable of controlling the concentrations of said at least one chemical in said bath, where said third data signal is transmitted to said controller and where said third data signal is used by said controller to control said concentration; and
d) a communication protocol selected from the group consisting essentially of an industrial ethernet, a TCP/IP protocol, a NetDDE, an OPC server, or a combination of the foregoing and wherein said controller and said analyzer are interfaced using said communication protocol.

34. The system of claim 33, wherein the controller controls the analyzer by transmitting a fourth data signal having an open communication protocol to the second PC and where a combination of hardware and software enables the second PC to translate the fourth data signal to a fifth data signal having a proprietary communication protocol.

35. The system of claim 35, wherein the fifth data signal is transmitted to the first computer and wherein the first computer transmits a sixth data signal to the analyzer, said sixth signal having a proprietary communication protocol.

36. The system of claim 35, wherein the sixth data signal initiates measurement of the concentrations of one or more chemicals in said bath.

37. The system of claim 33, wherein said RTA and controller are interfaced utilizing a multi-layer communication model, wherein the multi-layer communication model comprises:
a) a data link layer comprising hardware for establishing physical connection between the analyzer and controller;
b) a network layer comprising software for routing data from node to node;
c) a transport layer comprising software for performing error checking and/or controlling transmission; and
d) a presentation layer comprising software for converting data in a first form into data in a second form for transmission, and then converting data that is transmitted in the second form back to the first form for additional transmission.

38. The system of claim 37, wherein data is converted into ASCII format for transmission in the presentation layer.

39. The system of claim 37, further comprising a session layer comprising a LAN for creating and maintaining communication channels.

40. The system of claim 37, further comprising a mathematical algorithm for estimating target chemical concentrations at a given time, said algorithm comprising equations for a time update and a measurement update, wherein said mathematical algorithm is utilized by said controller.

41. The system of claim 40, wherein the estimated target chemical concentration is compared with actual measured concentration and wherein any difference between said concentrations causes a calculation of a potential and/or actual dose of a chemical for maintaining the predetermined chemical concentration in said bath.

42. The system of claim 37, further comprising an application layer, where said application layer comprises a user interface program used by the RTA computer.

43. The system of claim 42, wherein the RTA user interface program is coupled with an application to serve up data to the IPC.

44. The system of claim 43, wherein the server application is installed on the RTA PC and the program and server application exchange data via DDE or ActiveX.

45. The system of claim 42, wherein the application is installed on the IPC and the program and server application exchange data via NetDDE or DCOM.

46. A method of maintaining a predetermined concentration of one or more chemicals in a liquid bath during manufacturing, said method comprising:
a) predetermining an acceptable concentration range for at least one chemical used in said bath during manufacturing;
b) predicting a depletion in concentration of at least one chemical that will occur during manufacturing;
c) using a controller to implement dosing of at least one chemical into the bath to correct the predicted depletion in concentration amounts to maintain the acceptable concentration range;
d) using a real time analyzer to obtain a measurement of real time concentration of at least one chemical in the bath;
e) transmitting the measured real time concentration to a controller;
f) using a controller to compare said real time concentration with the predicted concentration range and determining any deviation between said concentrations; and
g) using the controller to determine and/or implement dosing amounts to be added to the bath to maintain the acceptable concentration range.

47. The method of claim 46, further comprising the step of adjusting the chemistry of the bath when the deviation between said concentrations exceeds a predetermined amount.

48. The method of claim 47, wherein the controller is used to predict and/or correct the concentration of one or more chemicals in the bath between real time measurements.

49. The method of claim 48, wherein the controller is programmed with an algorithm to obtain the predicted concentration and/or concentration required for correction of the bath concentration, and wherein said algorithm is written into a program development language and compiled into an executable format.

50. The method of claim 46, wherein the controller maintains the concentration range by implementing dosing of one or more chemicals into the bath and/or draining a portion of the bath to maintain the acceptable concentration.
